(19) United States
(12) Patent Application Publication
(10) Pub. No.: US 2016/0058503 A1
(43) Pub. Date: Mar. 3, 2016

(54) METHODS FOR MODULATING RENAL NERVE TISSUE AND ASSOCIATED SYSTEMS AND DEVICES

(71) Applicant: Medtronic Ardian Luxembourg S.a.r.I., Luxembourg (LU)

(72) Inventors: Stefan Tunev, Santa Rosa, CA (US); Jennifer Ruwart, Grand Blanc, MI (US); Carol Sullivan, Santa Rosa, CA (US)

(21) Appl. No.: 14/839,893

(22) Filed: Aug. 28, 2015

Related U.S. Application Data
(60) Provisional application No. 62/042,826, filed on Aug. 28, 2014.

(51) Int. Cl. A61B 18/14; A61N 1/36

(52) U.S. Cl. CPC A61B 18/1492; A61N 1/36117; A61B 2018/00577

(57) ABSTRACT

Methods for treating a patient using therapeutic renal neuromodulation and associated devices, system, and methods are disclosed herein. One aspect of the present technology is directed to neuromodulating nerve tissue in selected anatomical regions. In one embodiment, the method can include intravascularly positioning a neuromodulation element of a catheter within renal vasculature of a human patient and modulating nerve tissue within an anatomical region extending circumferentially around a branch vessel along a proximal-most longitudinal length of the branch vessel (e.g., between about 1 mm to about 12 mm distal to a bifurcation). The method can also include positioning the neuromodulation element within a second branch vessel and modulating nerve tissue within an anatomical region extending circumferentially around the second branch vessel along a proximal-most longitudinal length of the second branch vessel (e.g., between about 1 mm to about 12 mm distal to a bifurcation).

### CROSS-REFERENCE TO RELATED APPLICATION(S)

This application claims the benefit of U.S. Provisional Patent Application No. 62/042,826, filed Aug. 28, 2014, and incorporated herein by reference in its entirety.

### TECHNICAL FIELD

The present technology is related to neuromodulation, such as renal neuromodulation and systems, devices, and methods for performing renal neuromodulation on human patients.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS, in particular, has been identified experimentally and in humans as a likely contributor to the complex pathophysiologies of hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome. Pharmacologic strategies to mitigate adverse consequences of renal sympathetic stimulation often include the use of centrally-acting sympatholytic drugs, beta blockers, angiotensin-converting enzyme inhibitors, and/or diuretics. These and other pharmacologic strategies, however, tend to have significant limitations including limited efficacy, compliance issues, and undesirable side effects.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIG. 1 is a partially cross-sectional profile view illustrating a neuromodulation element (shown schematically) of a catheter delivering energy to nerve tissue within an anatomical region extending circumferentially around a distalmost portion of a main vessel of renal vasculature of a human patient in accordance with an embodiment of the present technology.

FIGS. 1A-1D are histological images of stained tissue slices from cross-sections of renal tissue of a pig and showing the proximity of the nerve fibers 124 along portions of the renal vasculature shown in FIG. 1.

FIG. 2 is a partially cross-sectional profile view illustrating a neuromodulation element (shown schematically) of a catheter delivering energy to nerve tissue within an anatomical region extending circumferentially around a portion of a branch vessel of the renal vasculature in accordance with an embodiment of the present technology.

FIG. 3 is a flow chart illustrating a renal neuromodulation method in accordance with an embodiment of the present technology.

FIG. 4 is a partially cross-sectional profile view illustrating a first sheath and a first catheter within the renal vasculature in accordance with an embodiment of the present technology. As shown in FIG. 4, the first catheter can include a first shaft and a first neuromodulation element. The first neuromodulation element can include a first balloon. In FIG. 4, the first neuromodulation element is in an expanded treatment state within the distalmost portion of the main vessel.

FIG. 5 is a partially cross-sectional profile view illustrating a second sheath and a second catheter within the renal vasculature in accordance with an embodiment of the present technology. As shown in FIG. 5, the second catheter can include a second shaft and a second neuromodulation element. The second neuromodulation element can include a second balloon. In FIG. 5, the second neuromodulation element is in a low-profile delivery state within a branch vessel of the renal vasculature.

FIG. 6 is an enlarged, partially cross-sectional profile view illustrating the second sheath and the second catheter of FIG. 5 within the renal vasculature. In FIG. 6, the second neuromodulation element is in an expanded treatment state within the branch vessel.

FIG. 7 is a flow chart illustrating a renal neuromodulation method in accordance with an embodiment of the present technology.

FIG. 8 is a partially cross-sectional profile view illustrating a sheath within the renal vasculature in accordance with an embodiment of the present technology. A catheter (not shown in FIG. 8) is disposed within the sheath. The catheter can include a first neuromodulation element and a second neuromodulation element in respective low-profile delivery states within the main vessel.

FIG. 9 is a partially cross-sectional profile view illustrating the sheath and the catheter of FIG. 8 within the renal vasculature in accordance with an embodiment of the present technology. As shown in FIG. 9, the first neuromodulation element is deployed from the sheath and in an expanded treatment state within the distalmost portion of the main vessel and the second neuromodulation element is deployed from the sheath and in its low-profile delivery state within the distalmost portion of the main vessel.

FIG. 10 is a partially cross-sectional profile view illustrating the sheath and the catheter of FIG. 8 within the renal vasculature in accordance with an embodiment of the present technology. As shown in FIG. 10, the second neuromodulation element is deployed from the sheath and in its low-profile delivery state within the main vessel. The first neuromodulation element (not shown in FIG. 10) is also in its low-profile delivery state within the main vessel.

FIG. 11 is a partially cross-sectional profile view illustrating the sheath and the catheter of FIG. 8 within the renal vasculature in accordance with an embodiment of the present technology. As shown in FIG. 11, the second neuromodulation element is deployed from the sheath and in its low-profile delivery state within a branch vessel. The first neuromodulation element (not shown in FIG. 11) is in its low-profile delivery state within the main vessel.

FIG. 12 is an enlarged, partially cross-sectional profile view illustrating the sheath and the catheter of FIG. 8 within the renal vasculature in accordance with an embodiment of the present technology. As shown in FIG. 12, the second neuromodulation element is in an expanded treatment state within the branch vessel. The first neuromodulation element (not shown in FIG. 12) is its low-profile delivery state within the main vessel.

FIG. 13 is a perspective view illustrating a neuromodulation system configured in accordance with an embodiment of the present technology.

FIG. 14 is a plot of mean norepinephrine concentration before and after renal neuromodulation procedures performed at different treatment sites within the renal vasculatures of animal subjects.

FIG. 15A is another plot of mean norepinephrine concentration before and after renal neuromodulation procedures performed in the distal portions of the main renal artery, the renal arterial branches, and the combination of the distal portions of the main renal artery and the renal arterial branches of animal subjects.

FIG. 15B is a plot of terminal axon density in the renal cortex corresponding to the treatment sites within the renal vasculature of the animal subjects of FIG. 15A.

FIG. 16A is a display table illustrating results from a study to determine the effects of renal denervation on cortical axon density and mean norepinephrine concentration in animal subjects.

FIG. 16B is a series of graphs illustrating the response correlation between normalized cortical axon area vs. norepinephrine concentration and norepinephrine concentration vs. extent of nerve ablation along the artery of the animal subjects of FIG. 16A.

FIG. 17 is a line graph illustrating the mean distance (mm) with standard deviation between an arterial lumen and sympathetic nerves in proximity to and extending along the length of the renal vasculature in a representative human subject.

### DETAILED DESCRIPTION

The present technology is related to neuromodulation, such as renal neuromodulation, and systems, devices, and methods for performing renal neuromodulation on human patients. The inventors have discovered, among other things, that targeting certain locations within a patient's renal vasculature may increase the efficacy of renal neuromodulation for achieving one or more desired clinical outcomes, such as lowering of a patient's blood pressure. Renal neuromodulation treatments can include, for example, targeting one or more anatomical regions of the patient's renal vasculature, and can include a combination of treating one or more proximal and/or central portions of the main artery, one or more distal portions of the main artery, one or more branch vessels, and/or at one or more bifurcations of the renal vasculature. A renal neuromodulation method in accordance with a particular embodiment of the present technology includes preferentially targeting nerve tissue for treatment within an anatomical region extending circumferentially around a distalmost portion (e.g., a distalmost third, quarter, or other suitable fraction) of a main vessel of a patient's renal vasculature. In addition or alternatively, the method can include preferentially targeting nerve tissue for treatment within an anatomical region extending circumferentially around one or more branch vessels of a patient's renal vasculature. In further embodiments, the method can include targeting nerve tissue within an anatomical region extending circumferentially around one or more branch vessels and around one or more portions (e.g., a proximal portion, a central portion, a distalmost portion) of the main vessel of a patient's renal vasculature. In yet further embodiments, the method can include targeting and neuromodulating nerve tissue within an anatomical region extending circumferentially around a trunk segment of one or more branch vessels. For example, the trunk segment can span the longitudinal axis of the branch vessels from about 1 mm to about 6 mm distal to the bifurcation of the main renal vessel. Targeting nerve tissue for treatment within these anatomical regions may allow a neuromodulation procedure to reliably achieve relatively comprehensive renal neuromodulation, i.e., adequately treating all or nearly all of these nerve fibers innervating a kidney.

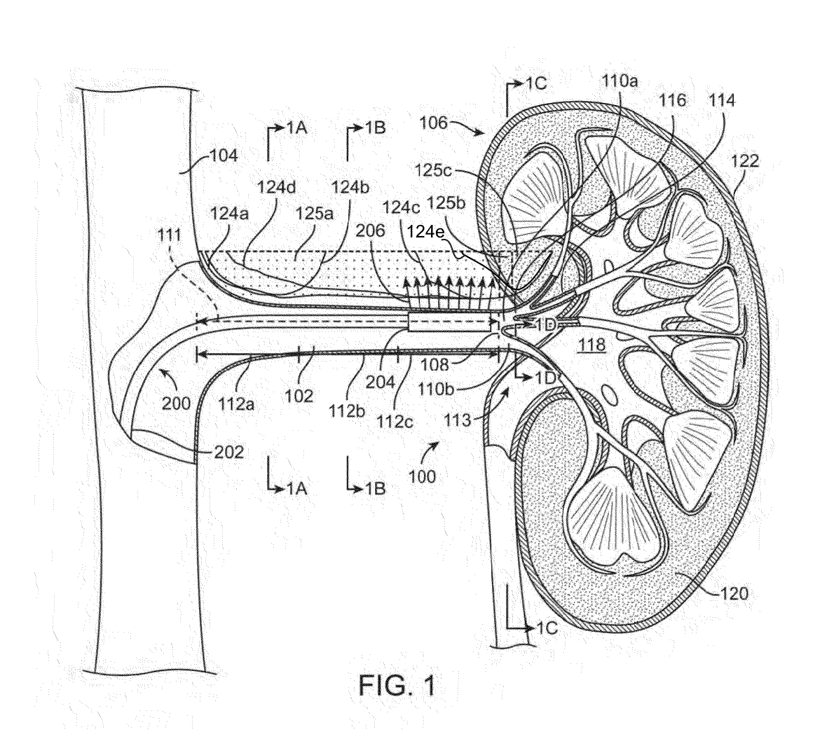

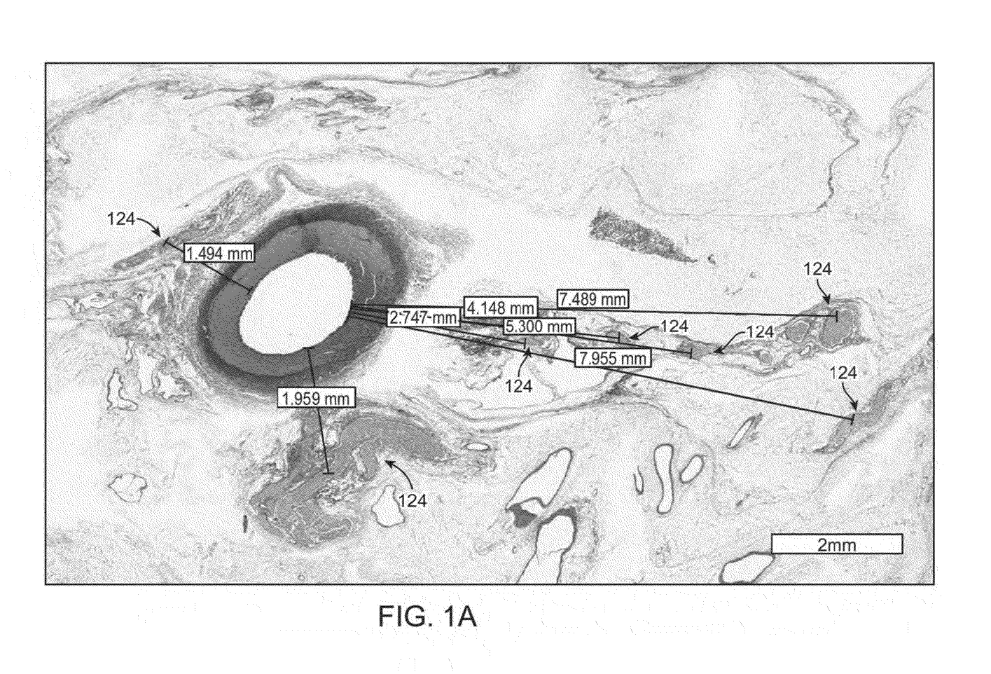

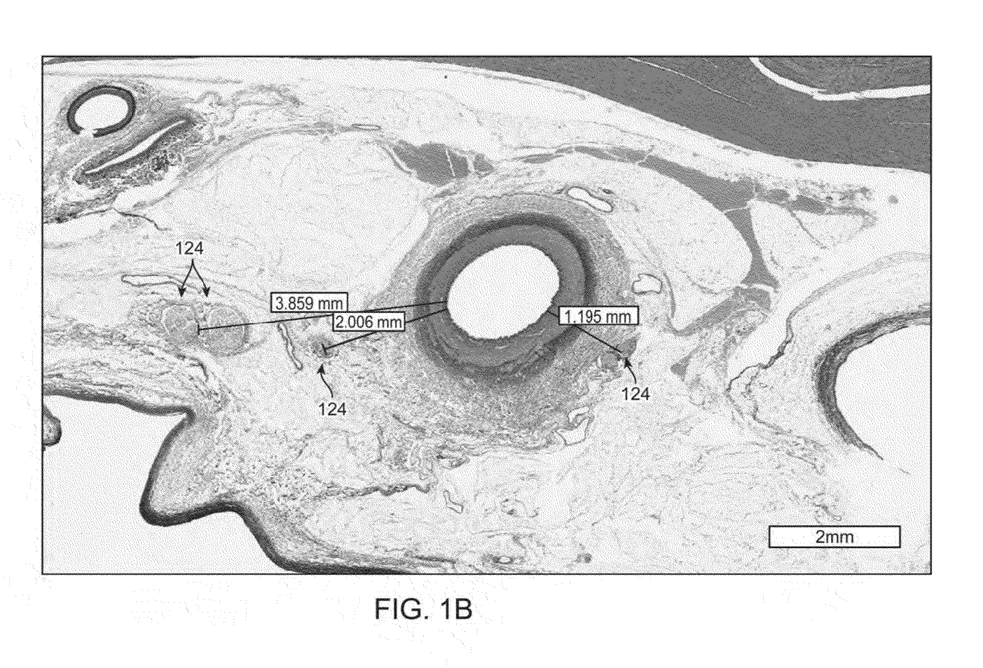

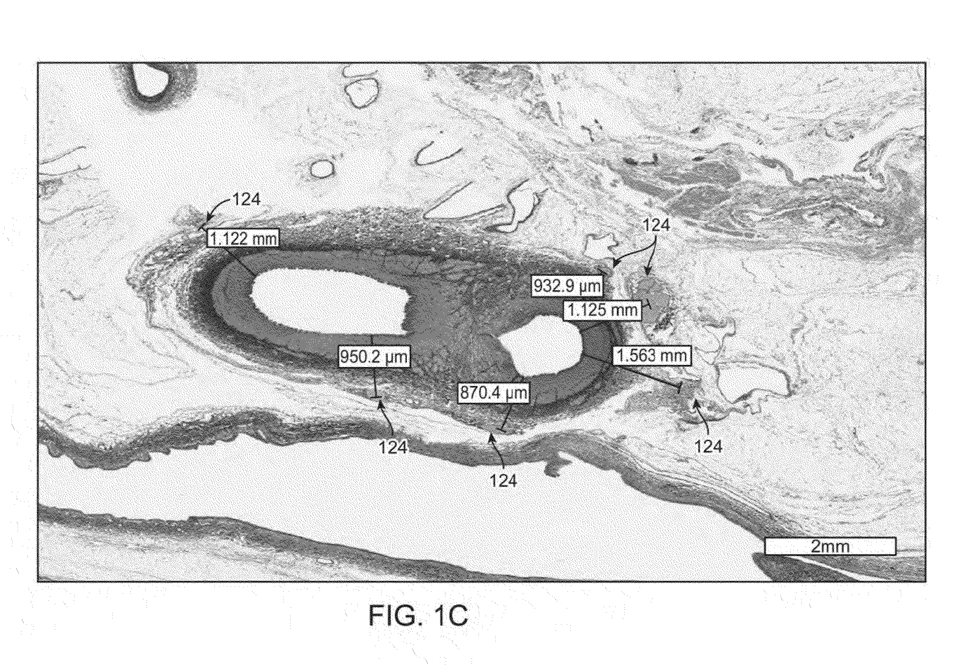

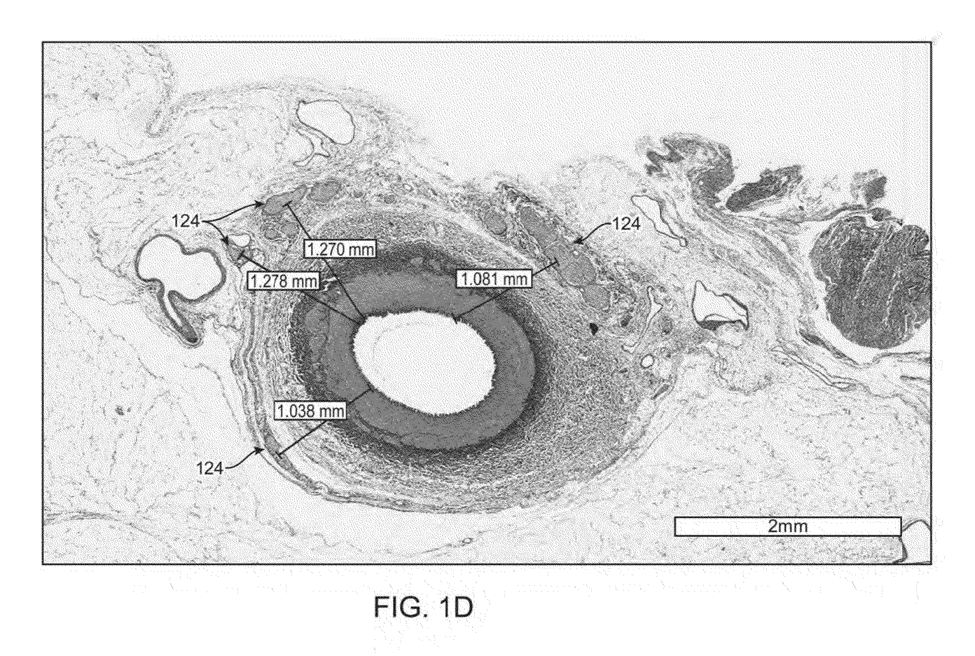

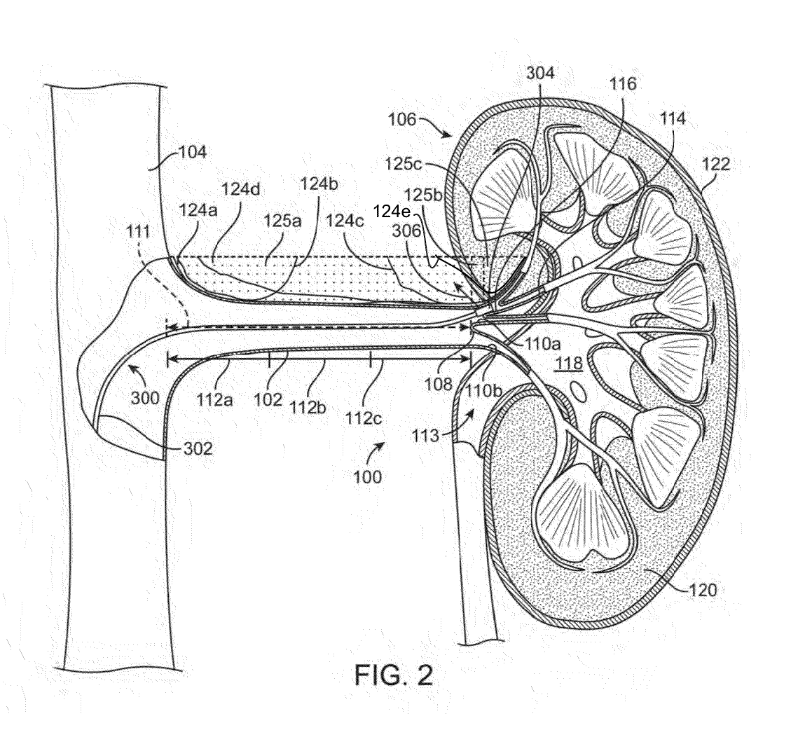

FIGS. 1 and 2 are partially cross-sectional profile views illustrating neuromodulation methods in accordance with embodiments of the present technology. FIGS. 1 and 2 also illustrate renal vasculature 100 of a human patient and associated anatomy. The renal vasculature 100 includes a main vessel 102 (e.g., a main artery) directly connected to an aorta 104 and extending distally toward a kidney 106. The renal vasculature 100 further includes a primary bifurcation 108 at a distal end of the main vessel 102, a first primary branch vessel 110a extending distally from the main vessel 102 beginning at the primary bifurcation 108, and a second primary branch vessel 110b extending distally from the main vessel 102 also beginning at the primary bifurcation 108. The main vessel 102 has a longitudinal axis 111 and a corresponding length 112 extending distally from the aorta 104 to the primary bifurcation 108. Distal to the first and second primary branch vessels 110a, 110b, the renal vasculature 100 includes a network 113 of subordinate branch vessels 114 and subordinate bifurcations 116. For clarity of illustration only one subordinate branch vessel 114 and one subordinate bifurcation 116 are identified in FIGS. 1 and 2. From the subordinate branch vessels 114, the network 113 branches into capillaries (not shown) that supply blood to the kidney 106. The first and second primary branch vessels 110a, 110b can be, for example, segmental arteries. The subordinate branch vessels 114 can be, for example, segmental arteries, interlobular arteries, and/or arcuate arteries. Collectively, the first primary branch vessel 110a, the second primary branch vessel 110b, and the subordinate branch vessels 114 are referred to herein as the branch vessels 110a, 110b, 114.

The kidney 106 includes a pelvis 118 and a cortex 120 extending around the pelvis 118. Blood flows into the kidney 106 through arteries of the renal vasculature 100 via the pelvis 118 and flow out of the kidney 106 through veins (not shown) of the renal vasculature 100 also via the pelvis 118. The kidney 106 further includes a capsule 122 encasing the cortex 120. The capsule 122 may preclude passage of nerve tissue. Thus, all or substantially all renal neural communication follows the renal artery and flows into and out of the kidney 106 through the renal pelvis 118. Several examples of nerve fibers 124 are shown in FIGS. 1 and 2. For clarity of illustration, the nerve fibers 124 are shown in FIGS. 1 and 2 in two dimensions and extending through a first anatomical region 125a adjacent to the main vessel 102, a second anatomical region 125b adjacent to the first primary branch vessel 110a, and a third anatomical region 125c adjacent to one of the subordinate branch vessels 114. Also for clarity of illustration, the nerve fibers 124 are shown as single fibers; however, it is understood that nerves are typically arranged in nerve bundles each having more than one nerve fiber along the first, second and third anatomical regions 125a-c. It should be understood that the first, second, and third anatomical regions 125a-125c extend circumferentially in three dimensions around the main vessel 102, the first primary branch vessel 110a, and the subordinate branch vessel 114, respectively. It should also be understood that the anatomy associated with the renal vasculature 100 includes other anatomical regions extending circumferentially in three dimensions around the second primary branch vessel 110b and additional subordinate branch vessels 114, respectively, and that the nerve fibers 124 can be distributed at various locations in three dimensions within the first, second, and third anatomical regions 125a-125c and within these other anatomical regions. Collectively, the first, second, and third anatomical regions 125a-125c and these other anatomical regions are identified herein as the anatomical regions 125. As shown in FIGS. 1 and 2, the anatomical regions 125 can include tissue outside the capsule 122 as well as tissue inside the capsule 122.

The nerve fibers 124 bifurcate at or near the primary and subordinate bifurcations 108, 116 and follow the subordinate branch vessels 114 fairly closely within the cortex 120. The nerve fibers 124 eventually terminate at various levels of the renal arterial tree up to and including the afferent arterioles where they can control vasodilation and vasoconstriction. In some cases, all of the nerve fibers 124 also follow the main vessel 102 fairly closely within a well-defined plexus and extend along the entire length 112 of the main vessel 102 or nearly the entire length 112 of the main vessel 102 at a relatively uniform distance from a wall of the main vessel 102. In these cases, a neuromodulation treatment at any portion of the first anatomical region 125a is expected to be efficacious.

By way of theory (and without wishing to be bound by theory), in certain other cases only some of the nerve fibers 124 (e.g., the nerve fiber 124a) are of a first type that extends along the entire length 112 of the main vessel 102 or nearly the entire length 112 of the main vessel 102 at a relatively uniform distance from a wall of the main vessel 102. As mentioned above, a renal neuromodulation treatment performed at any position along the length 112 of the main vessel 102 may be relatively effective for treating this first type of nerve fiber 124. Other nerve fibers 124 (e.g., the nerve fiber 124b), however, may be of a second type extending along a proximal part of the length 112 of the main vessel 102 and then diverging from the main vessel 102 toward a non-renal destination. Further, other nerve fibers 124 (e.g., the nerve fiber 124c) may be of a third type that approaches the wall of the main vessel 102 abruptly at a relatively distal position along the length 112 of the main vessel 102. Still further, other nerve fibers 124 (e.g., the nerve fiber 124d) may be of a fourth type that approaches the wall of the main vessel 102 gradually along the length 112 of the main vessel 102 from proximal to distal. Other types of nerve fibers 124 are also possible. For example, nerve fibers 124 (e.g., the nerve fiber 124e) may approach a wall of a branch artery distal to the primary bifurcation 108. Accordingly, nerve fibers 124e of this fifth type may not be accessible for treatment along the main vessel 102. Renal neuromodulation treatment performed within one or more branch vessels 110a, 110b, 114 may be relatively effective for treating this fifth type of nerve fiber 124e.

Renal neuromodulation treatments performed at certain positions along the length 112 of the main vessel 102 may have advantages relative to renal neuromodulation procedures performed at other positions along the length 112 of the main vessel 102. For example, a renal neuromodulation treatment performed from within the main vessel 102 at a relatively distal position along the length 112 of the main vessel 102 and/or from within one or more of the branch vessels 110a, 110b, 114 may avoid treating the second type of nerve fibers 124 (e.g., the nerve fiber 124b) unnecessarily. This can be useful, for example, because nerve fibers 124 of the second type do not terminate within the kidney 106. As another example, a renal neuromodulation treatment performed from within the main vessel 102 at a relatively distal position along the length 112 of the main vessel 102 and/or from within one or more of the branch vessels 110a, 110b, 114 may be well-suited for treating the third type of nerve fibers 124 (e.g., the nerve fiber 124c), and/or the fifth type of nerve fibers 124 (e.g., the nerve fiber 124e) such as by delivering energy distal to where the nerve fibers 124 join the path of the main vessel 102 or the branch vessels 110a, 110b and 114.

As yet another example, a renal neuromodulation treatment performed from within the main vessel 102 at a relatively distal position along the length 112 of the main vessel 102 and/or from within one or more of the branch vessels 110a, 110b, 114 may be well-suited for providing a therapeutically effective amount of energy to the fourth type of nerve fibers 124 (e.g., the nerve fiber 124d), such as by delivering energy distal to a point along the length 112 of the main vessel 102 at which the nerve fibers 124 begin to travel along the length 112 of the main vessel 102 in close enough proximity to a wall of the main vessel 102 to be within a therapeutically effective range of a neuromodulation element. For example, in portions of the renal vasculature, a greater number of nerve fibers 124 are accessible (e.g., within range) to the neuromodulating treatment. FIGS. 1A-1D are cross-sectional histological images taken from stained tissue slices from renal tissue of a pig and showing the proximity of the nerve fibers 124 (stained with Movat's trichrome) along portions of the renal vasculature shown in FIG. 1. FIG. 1A shows the proximity of nerve fibers 124 to an inner wall of the proximal portion of the main renal artery and FIG. 1B shows the proximity of nerve fibers 124 to the inner wall of the central/middle portion of the main renal artery. FIG. 1C shows the proximity of nerve fibers 124 to an inner wall of branch vessels at the bifurcation of the main renal artery and FIG. 1D shows the proximity of nerve fibers 124 to an inner wall of a branch vessel distal to the bifurcation (e.g., approximately 3-4 mm distal to the bifurcation). Referring to FIGS. 1A-1D together, the nerve fibers 124 are shown to be closer in proximity (e.g., more accessible to treatment originating from the inner lumen of the renal arterial vasculature) at the bifurcation and branch vessels as compared to the proximal and central/middle portions of the main renal artery. Accordingly, systems, devices, and methods for performing renal neuromodulation in accordance with embodiments of the present technology are expected to be well-suited for effectively treating the first, third, fourth, and fifth types of nerve fibers 124, while avoiding unnecessary treatment of the second type of nerve fibers 124.

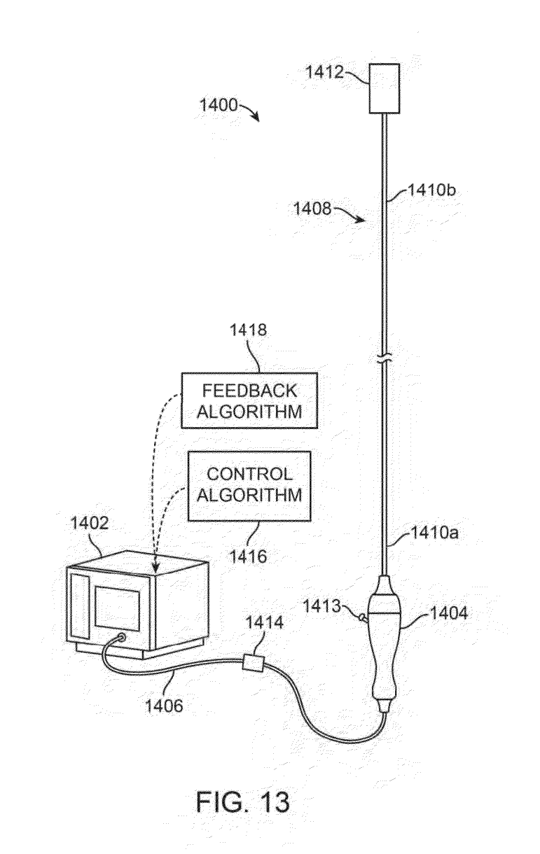

In a further example, the therapeutic energy (e.g., radiofrequency (RF) energy) can be delivered at different levels (e.g., intensities, power levels) at varying positions along the length 112 of the main vessel 102 and/or the branches. For example, in regions of the renal vasculature where the nerve fibers 124 are further from the inner wall of the vessel, the power may be increased. By increasing the power output from the electrode, the RF energy can increase the three-dimensional area of the resulting lesion. As such, the resultant larger lesion would reach greater tissue depths from the inner wall of the vessel. Likewise, where the nerve fibers 124 are found closer to the inner wall of the vessel, the power may be selectively decreased such that damage to non-target tissue is minimized while still achieving successful denervation. In some embodiments, the system (e.g., console 1402, discussed further with respect to FIG. 13 below) and/or electrodes spaced apart along a multi-electrode neuromodulation element, can be configured such that the electrodes individually supply different and/or varying amounts of energy (e.g., RF energy) based on the electrode's location along the vasculature. For example, the system can be configured such that an electrode positioned along the proximal portion of the main vessel 102 imparts higher power than an electrode positioned along the distalmost portion of the main vessel 102 and/or the branch vessels 110a, 110b, 114.

In further embodiments, the system and/or electrode(s) can be configured to vary the duration of power delivery either collectively or individually (e.g., in embodiments having multi-electrode neuromodulation elements). In various arrangements, the duration of power delivery can vary depending on the position of one or more electrodes along the vasculature. For example, the system can be configured such that an electrode positioned along the proximal portion of the main vessel 102 imparts power for a longer duration than an electrode positioned along the distalmost portion of the main vessel 102 and/or the branch vessels 110a, 110b, 114. In a particular embodiment, for example, the electrodes spaced apart along a multi-electrode neuromodulation element can be controlled to selectively deliver power at individually selected power levels and for individually selected durations such that power delivery is optimized for targeting nerve tissue at varying depths along the renal vasculature.

In yet further examples, renal neuromodulation treatments can be performed at certain positions along the length 112 of the main vessel 102, the branch vessels 110a, 110b, 114, or both in a patient-specific dependent manner. For example, a clinician can assess via angiogram, fluoroscope, etc., a patient's particular anatomy and disease state (e.g., stenosis, arthrosclerosis, vessel diameter, degree of vessel torsion, vessel length, branch length distal to the bifurcation, etc.) and determine one or more desirable locations for renal neuromodulation treatment.

The main vessel 102 may be stented or unstented during renal neuromodulation in accordance with at least some embodiments of the present technology. In one example, the main vessel 102 is stented in an earlier attempt to achieve a desired clinical outcome. Renal neuromodulation in accordance with an embodiment of the present technology may be used when stenting the main vessel 102 is not effective or is insufficiently effective for achieving the clinical outcome. For example, renal neuromodulation in accordance with an embodiment of the present technology may be used to supplement the therapeutic effect, if any, of stenting the main vessel 102 on lowering a patient's blood pressure. Alternatively or in addition, stenting the main vessel 102 and renal neuromodulation in accordance with an embodiment of the present technology may have different purposes. Typically, when present, a renal stent (not shown) is located at a proximal portion of the main vessel 102. From this position, the stent is unlikely to interfere with the methods illustrated in FIGS. 1 and 2. Thus, these methods and at least some other methods in accordance embodiments of the present technology may be more compatible with a stented main vessel 102 than at least some conventional counterparts.

Specific details of systems, devices, and methods in accordance with several embodiments of the present technology are disclosed herein with reference to FIGS. 1-15B. Although the systems, devices, and methods may be disclosed herein primarily or entirely with respect to intra-arterial renal neuromodulation, other applications in addition to those disclosed herein are within the scope of the present technology. For example, systems, devices, and methods in accordance with at least some embodiments of the present technology may be useful for neuromodulation within one or more non-arterial or non-vessel body lumens, for extravascular neuromodulation, for non-renal neuromodulation, and/or for use in therapies other than neuromodulation. Furthermore, it should be understood, in general, that other systems, devices, and methods in addition to those disclosed herein are within the scope of the present technology. For example, systems, devices, and methods in accordance with embodiments of the present technology can have different and/or additional configurations, components, and procedures than those disclosed herein. Moreover, a person of ordinary skill in the art will understand that systems, devices, and methods in accordance with embodiments of the present technology can be without one or more of the configurations, components, and/or procedures disclosed herein without necessarily deviating from the present technology.

### Selected Examples of Neuromodulation Methods and Associated Technology

As shown in FIG. 1, a catheter 200 including an elongate shaft 202 and a neuromodulation element 204 (shown schematically) operably connected to the shaft 202 can be located such that the neuromodulation element 204 is distally positioned along the length 112 of the main vessel 102. In the illustrated embodiment, for example, the neuromodulation element 204 is located at least predominantly within a distal portion of the main vessel 102. Portions of the length 112 of the main vessel 102 corresponding to a distalmost third 112a, a middle third 112b, and a proximal-most third 112c of the main vessel 102 are respectively indicated in FIGS. 1 and 2.

As shown in FIG. 2, a catheter 300 including an elongate shaft 302 and a neuromodulation element 304 (shown schematically) operably connected to the shaft 302 can be located such that the neuromodulation element 304 is within the first primary branch vessel 110a. In the illustrated embodiment, the neuromodulation element 304 partially extends into one of the subordinate branch vessels 114. In other embodiments, however, the neuromodulation element 304 can be entirely within the first primary branch vessel 110a or partially extended into more than one of the subordinate branch vessels 114 and/or into the main vessel 102.

With reference to FIGS. 1 and 2 together, the neuromodulation elements 204, 304 can be more longitudinally compact than at least some conventional counterparts and can be positioned at respective treatment sites within the renal vasculature 100 relatively well-suited for comprehensively treating the nerve fibers 124. From its position shown in FIG. 1, the neuromodulation element 204 can be used to deliver energy (represented by arrows 206) to the nerve fibers 124 within a portion of the first anatomical region 125a extending around the distal portion of the main vessel 102. From its position shown in FIG. 2, the neuromodulation element 304 can be used to deliver energy (represented by arrows 306) to the nerve fibers 124 within a portion of the second anatomical region 125b extending around the first primary branch vessel 110a and within a portion of the third anatomical region 125c extending around one of the subordinate branch vessels 114. Typically, all or substantially all nerve fibers 124 targeted for treatment (e.g. nerve fibers 124 of the first, third, fourth, and fifth types discussed above, among others) are reliably found in close proximity to the renal vasculature 100 within the anatomical region 125 immediately distal and proximal to the primary bifurcation 108.

In FIG. 1, energy from the neuromodulation element 204 is shown extending in one planar direction toward the first anatomical region 125a. Similarly, in FIG. 2, energy from the neuromodulation element 304 is shown extending in one planar direction toward the second anatomical region 125b. These are simplified representations merely for clarity of illustration. Instead of extending in one planar direction, energy from the neuromodulation element 204 generally extends in many planar directions radially distributed about the longitudinal axis 111 of the main vessel 102. Similarly, energy from the neuromodulation element 304 generally extends in many planar directions radially distributed about a longitudinal axis (not shown) of the first primary branch vessel 110a.

In some embodiments, it may be desirable to avoid delivering energy in a pattern that causes a circumferentially continuous lesion to form within any plane perpendicular to the longitudinal axis 111 of the main vessel 102 or a longitudinal axis of any of the branch vessels 110a, 110b, 114. Such a lesion is thought to potentially increase the risk of stenosis. For this and/or other reasons relating to vessel wall preservation, it may be desirable to deliver energy to the anatomical regions 125 in a helical/spiral pattern. Beyond the wall of a vessel from which the energy is delivered, different portions of a lesion formed in this manner may expand toward one another while still remaining circumferentially discontinuous within any plane perpendicular to the longitudinal axis of the vessel. If the nerve fibers 124 extend parallel to the longitudinal axis of the vessel and the sum of different portions of the lesion along the longitudinal axis of the vessel extends around the entire circumference of the vessel, then such a lesion is expected to reach all or substantially all of the nerve fibers 124. In some cases, however, the nerve fibers 124 may not extend parallel to the longitudinal axis of the vessel. Instead, the individual nerve fibers 124 may be arborized, interwoven, or otherwise irregular in their respective paths through the anatomical regions 125. Accordingly, when a helical/spiral lesion extends over a relatively large portion of the length of the vessel, some of the nerve fibers 124 may follow paths that avoid contact with any part of the lesion. Accordingly, the neuromodulation elements 204, 304 can be configured to form more longitudinally compact lesions than those formed by at least some conventional neuromodulation elements.

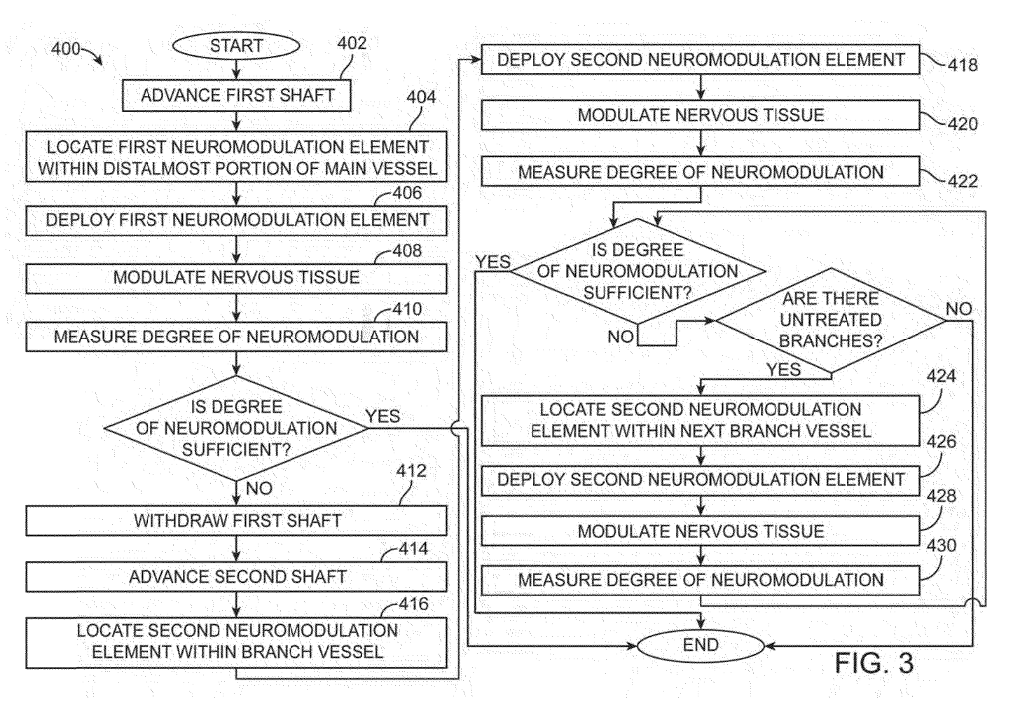

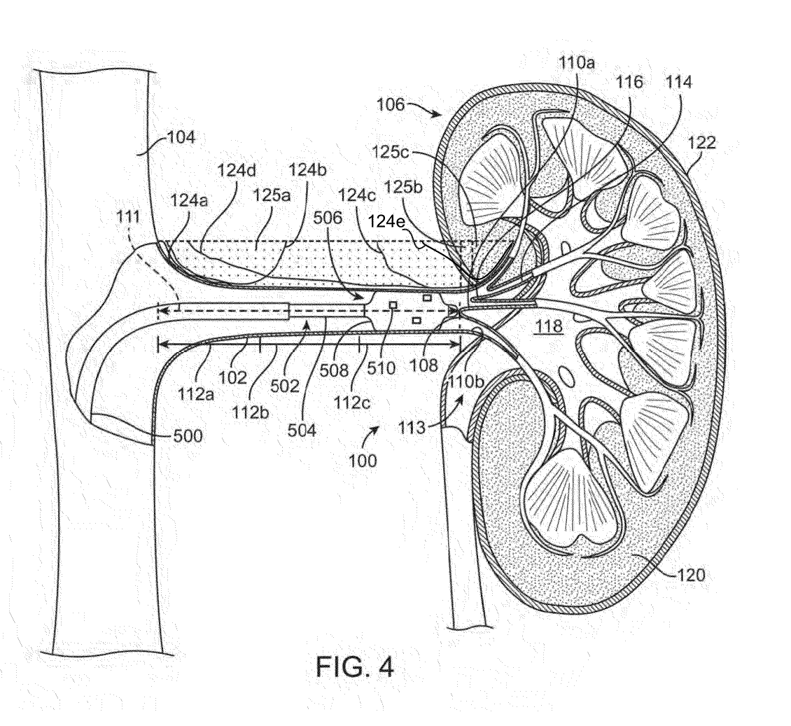

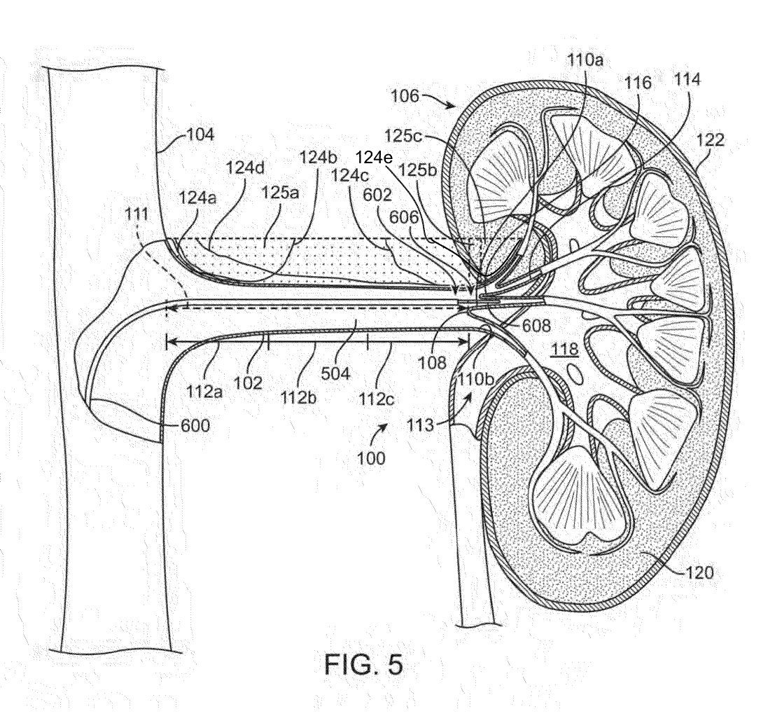

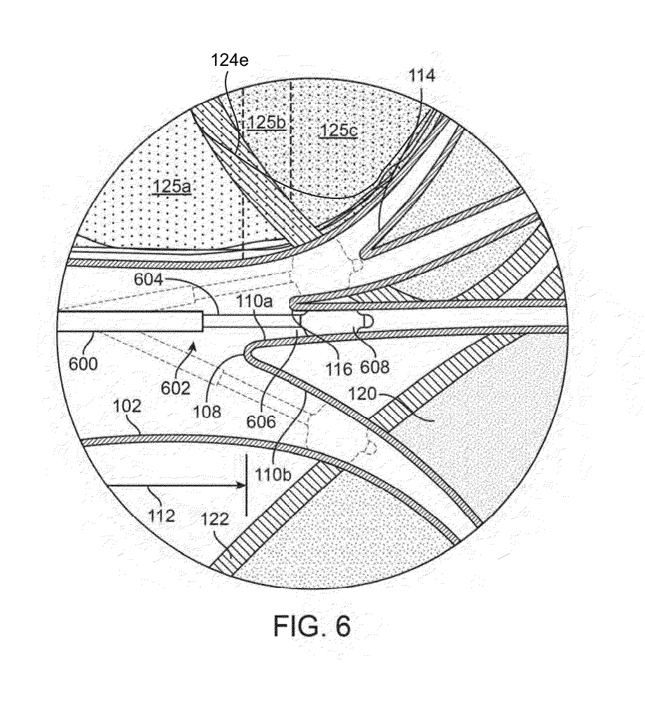

FIG. 3 is a flow chart illustrating a renal neuromodulation method 400 in accordance with an embodiment of the present technology. FIG. 4 is a partially cross-sectional profile view illustrating a first sheath 500 and a first catheter 502 within the renal vasculature 100 at one point during the method 400. As shown in FIG. 4, the first catheter 502 can include an elongate first shaft 504 and a first neuromodulation element 506 operably connected to the first shaft 504. FIG. 5 is a partially cross-sectional profile view illustrating a second sheath 600 and a second catheter 602 within the renal vasculature 100 at another point during the method 400. As shown in FIG. 5, the second catheter 602 can include an elongate second shaft 604 and a second neuromodulation element 606 operably connected to the second shaft 604. FIG. 6 is an enlarged, partially cross-sectional profile view illustrating the second sheath 600 and the second catheter 602 within the renal vasculature 100 at yet another point during the method 400.

With reference to FIGS. 3-6 together, the method 400 includes advancing the first shaft 504 intravascularly toward the renal vasculature 100, such as via a femoral, trans-radial or another suitable approach (block 402). While the first shaft 504 is advanced toward the renal vasculature 100, the first neuromodulation element 506 can be in a low-profile delivery state (not shown). For example, the first neuromodulation element 506 can include a first balloon 508, and the first balloon 508 can be deflated or otherwise unexpanded when the first neuromodulation element 506 is in the low-profile delivery state. After advancing the first shaft 504, the method 400 includes locating the first neuromodulation element 506 within the distal portion of the main vessel 102 (block 404). At this point, as shown in FIG. 4, the method 400 includes deploying the first neuromodulation element 506 into an expanded treatment state (block 406), such as by inflating or otherwise expanding the first balloon 508. The first neuromodulation element 506 can include first energy-delivery elements 510 (e.g., electrodes or transducers) spaced apart from one another and arranged in a helical/spiral pattern on an outside surface of the first balloon 508. As the first neuromodulation element 506 is deployed into the expanded treatment state, the first energy-delivery elements 510 move into contact with an inner surface of a wall of the main vessel 102.

Once the first neuromodulation element 506 is located within the distal portion of the main vessel 102 and in the expanded treatment state, the method 400 includes using the first neuromodulation element 506 to modulate nerve tissue within a portion of the first anatomical region 125a extending circumferentially around the distal portion of the main vessel 102 (block 408). The distal portion of the main vessel 102 can be, for example, a distalmost third of the main vessel 102, a distalmost quarter of the main vessel 102, a distalmost centimeter of the main vessel 102, or another suitable relatively distal portion of the main vessel 102. Modulating nerve tissue within the portion of the first anatomical region 125a extending circumferentially around the distal portion of the main vessel 102 can include, for example, preferentially modulating this nerve tissue relative to nerve tissue within portions of the first anatomical region 125a extending circumferentially around a proximal portion (e.g., a proximal-most third) and a middle portion (e.g., a middle third) of the main vessel 102. While some energy may be delivered to proximal or middle portions of the first anatomical region 125a, at least in the illustrated embodiment, the bulk of the energy released from the first neuromodulation element 506 is delivered to the distal portion of the first anatomical region 125a.

In at least some cases, the first neuromodulation element 506 is more longitudinally compact than conventional counterparts. For example, the first neuromodulation element 506 can be configured to form one or more lesions that extend through a wall of the main vessel 102 into the first anatomical region 125a along a helical/spiral path with relatively little distance (e.g., less than 4 millimeters on average) between neighboring turns. Once formed, the one or more lesions can be circumferentially continuous within the first anatomical region 125a along a plane perpendicular to a portion of the longitudinal axis 111 of the main vessel 102. The lesion(s) may extend through the distal portion of the main vessel 102 while still being circumferentially discontinuous at the wall of the main vessel 102 along all planes perpendicular to this portion of the longitudinal axis 111. This is expected to reduce or eliminate the possibility of the one or more lesions missing arborized nerve fibers 124 without causing undue risk of stenosis within the main vessel 102.

After using the first neuromodulation element 506, the method 400 includes measuring a first degree of neuromodulation achieved by using the first neuromodulation element 506 (block 410). Techniques for measuring the first degree of neuromodulation include measuring biomarkers, as further described in International Patent Application No. PCT/US2013/030041 (published as International Publication No. WO2013/134733 and titled “Biomarker Sampling in the Context of Neuromodulation Devices and Associated Systems and Methods”) or inoperatively monitoring nerve activity, as further described in International Patent Application No. PCT/IB2012/003055 and titled “Endovascular Nerve Monitoring Devices and Associated Systems and Methods”, both of which are incorporated herein by reference in their entireties. If the first degree of neuromodulation is sufficient (e.g., if the kidney 106 is at least substantially denervated), the method 400 can end. If the first degree of neuromodulation is not sufficient (e.g., if the kidney 106 is not at least substantially denervated), the method 400 includes withdrawing the first shaft 504 (block 412) and advancing the second shaft 604 intravascularly toward the renal vasculature 100 (block 414), such as along a guide wire or a guide lumen (not shown) also used to advance the first shaft 504 toward the renal vasculature 100.

As shown in FIG. 5, while the second shaft 604 is advanced toward the renal vasculature 100, the second neuromodulation element 606 can be in a low-profile delivery state. For example, the second neuromodulation element 606 can include a second balloon 608, and the second balloon 608 can be deflated or otherwise unexpanded when the second neuromodulation element 606 is in the low-profile delivery state. After advancing the second shaft 604, the method 400 includes locating the second neuromodulation element 606 within one of the branch vessels 110a, 110b, 114 distal to the primary bifurcation 108 (block 416). As discussed above, the second neuromodulation element 606 can also extend partially into one or more other vessels within the renal vasculature 100. In at least some cases, it can be useful to avoid positioning the second neuromodulation element 606 in direct contact with the primary bifurcation 108 so as to reduce the likelihood that the primary bifurcation 108 will be damaged during energy delivery. In some embodiments, such damage may contribute to stenosis.

At this point, as shown in FIG. 6, the method 400 can include deploying the second neuromodulation element 606 into an expanded treatment state (block 418), such as by inflating or otherwise expanding the second balloon 608. The second balloon 608 can carry one or more second energy-delivery elements (not shown) (e.g., electrodes or transducers) arranged in a helical pattern. As the second neuromodulation element 606 is deployed into the expanded treatment state, the second energy-delivery elements move into contact with an inner surface of a wall of the branch vessel 110a, 110b, 114 in which the second neuromodulation element 606 is deployed. Next, the method 400 includes using the second neuromodulation element 606 to modulate nerve tissue within a portion of the second and/or third anatomical regions 125b, 125c extending circumferentially around the branch vessel 110a, 110b, 114 in which the second neuromodulation element 606 is deployed (block 420).

After using the second neuromodulation element 606, the method 400 includes measuring a second degree of neuromodulation achieved via the second neuromodulation element 606 (block 422). If the second degree of neuromodulation is sufficient, the method 400 can end. If the second degree of neuromodulation is not sufficient, however, and if there are untreated branch vessels 110a, 110b, 114, the method 400 includes locating the second neuromodulation element 606 within one of the untreated branch vessels 110a, 110b, 114 while the second neuromodulation element 606 is in the low-profile delivery state (block 424) and redeploying the second neuromodulation element 606 (block 426). Next, the method 400 includes using the second neuromodulation element 606 to modulate nerve tissue within a portion of the second and/or third anatomical regions 125b, 125c extending circumferentially around the branch vessel 110a, 110b, 114 in which the second neuromodulation element 606 is redeployed (block 428). The method 400 further includes measuring a degree of neuromodulation achieved by using the second neuromodulation element 606 (block 430). This process can continue until the measured degree of neuromodulation is sufficient or there are no more untreated branch vessel 110a, 110b, 114. In FIG. 6, treatment of two additional branch vessels 110a, 110b, 114 is shown in dashed lines. In some cases, each treated branch vessel 110a, 110b, 114 is independently connected to the main vessel 102. In other cases, multiple branch vessels 110a, 110b, 114 can be treated along a single pathway extending from the main vessel 102 to the kidney 106.

In the embodiment illustrated in FIGS. 3-6, one catheter (i.e., the first catheter 502) is used to treat the first anatomical region 125a and another catheter (i.e., the second catheter 602) is used to treat the second and/or third anatomical regions 125b, 125c. Further, while the embodiment illustrated in FIGS. 3-6 describe treating the first anatomical region 125a followed by treating the second and/or third anatomical regions 125b, 125c, it is understood that in other embodiments, the second and/or third anatomical regions 125b, 125c can be treated in a first instance (e.g., using the second catheter 602) or prior to treatment of the first anatomical region 125a. Optionally, a degree of neuromodulation can be assessed after treating the second and/or third anatomical regions 125b, 125c. In such embodiments, if the degree of neuromodulation is insufficient, the first anatomical region 125a can be treated (e.g., using the first catheter 502) following treatment of the second and/or third regions 125b, 125c. For example, in a specific example, the second catheter 602 can be used to treat the second and/or third anatomical region 125b, 125c (e.g., in one or more branch segments of the renal vasculature). If desirable, the first catheter 502 can be used subsequently to treat the first anatomical region 125a in a distalmost portion (e.g., a distal third of the main renal artery).

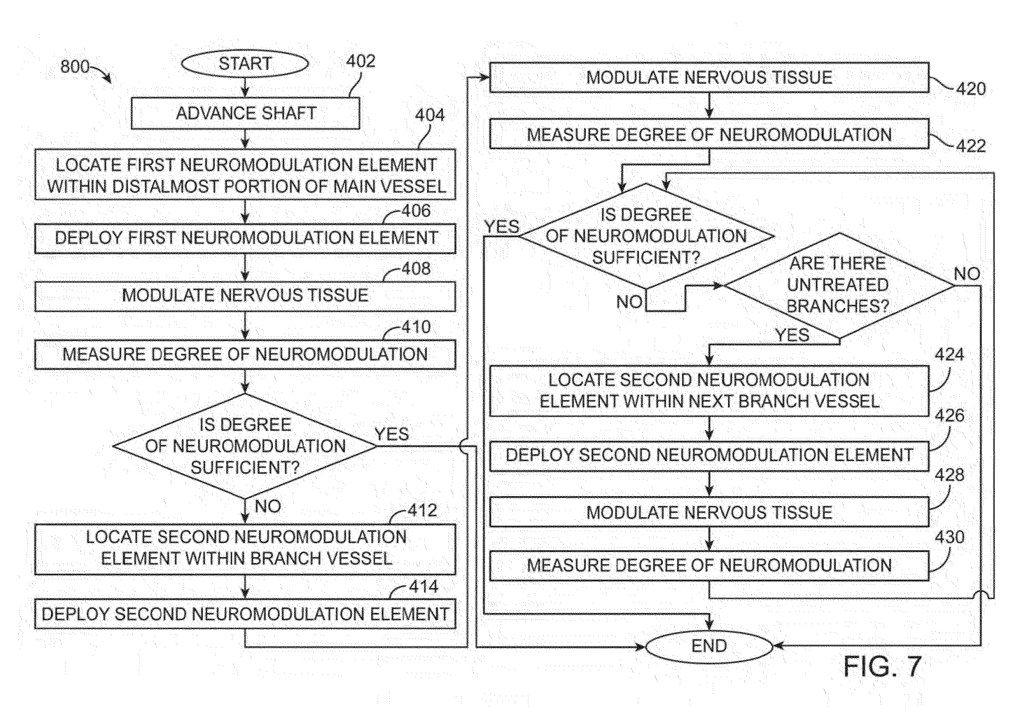

In other embodiments, a single catheter can be used to treat the first anatomical region 125a and the second and/or third anatomical regions 125b, 125c. FIGS. 7-12 illustrate such an embodiment. FIG. 7, for example, is a flow chart illustrating a renal neuromodulation method 800 in accordance with an embodiment of the present technology. The method 800 generally corresponds to the method 400 (FIG. 3) without the need to withdraw one shaft (e.g., the first shaft 504) and advance another shaft (e.g., the second shaft 604) if a first degree of neuromodulation is insufficient.

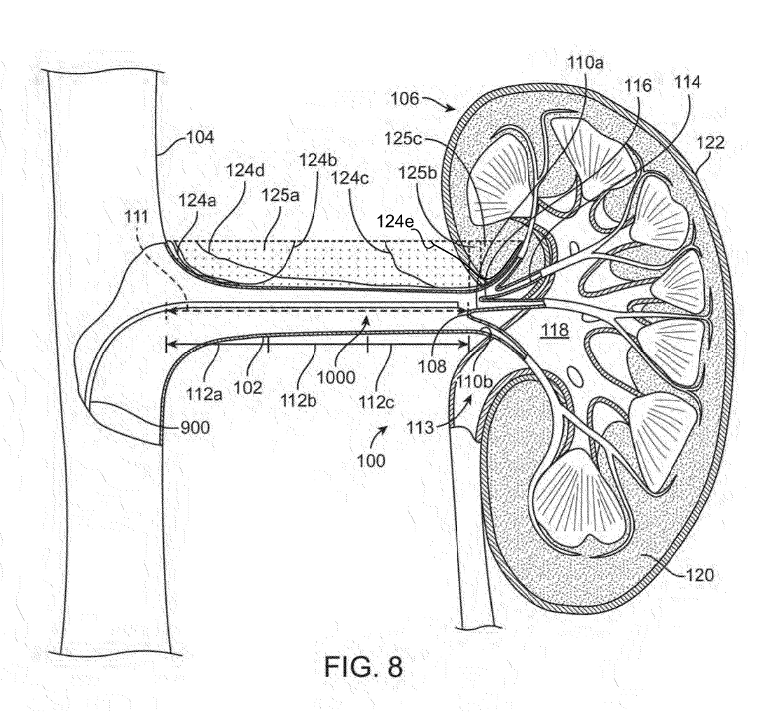

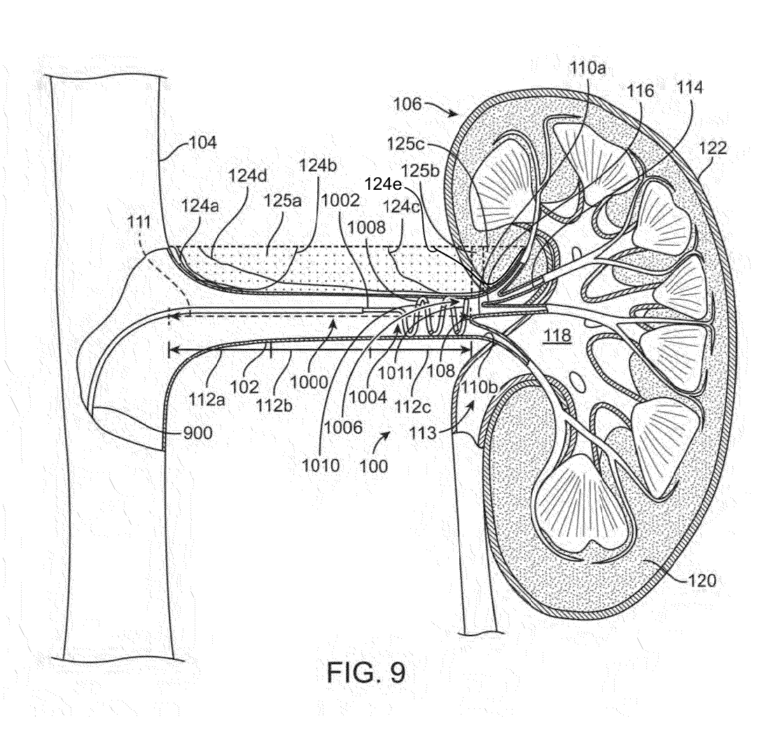

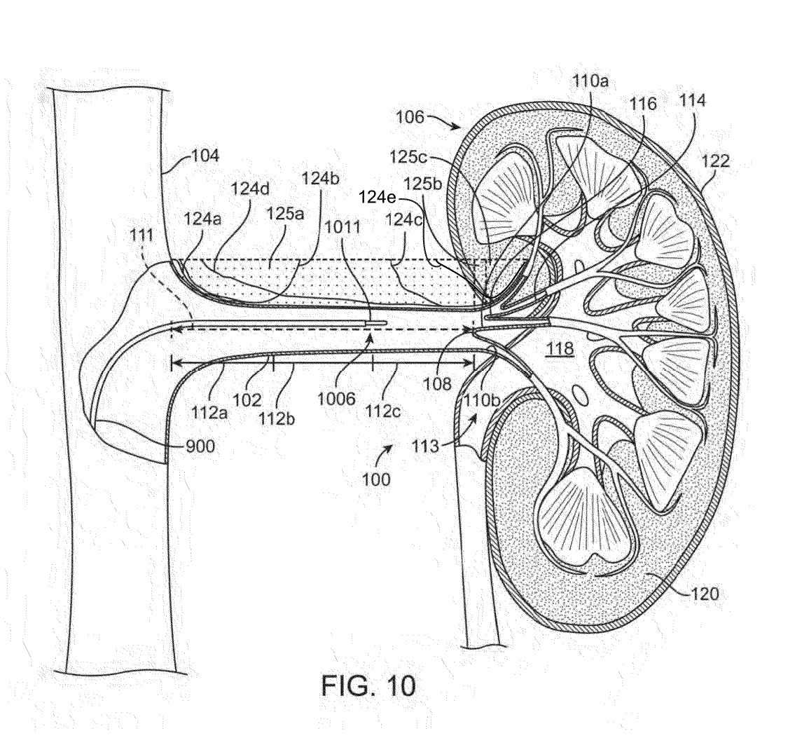

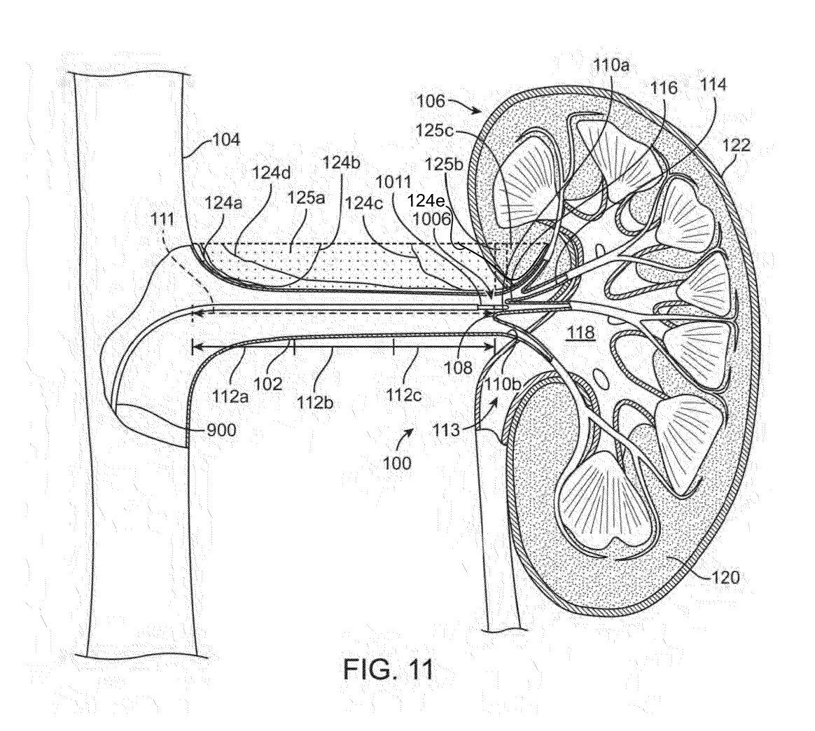

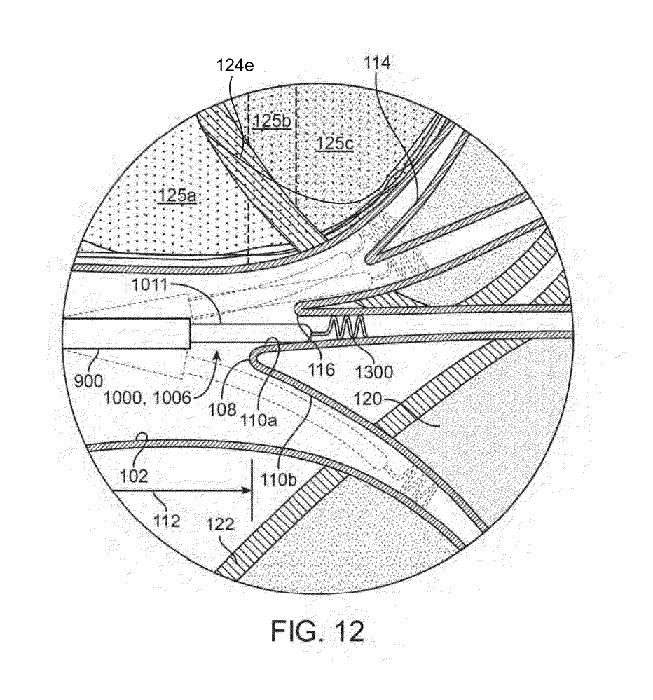

FIG. 8 is a partially cross-sectional profile view illustrating a sheath 900 within the renal vasculature 100 at one point during the method 800. FIG. 9 is a partially cross-sectional profile view illustrating the sheath 900 and a catheter 1000 within the renal vasculature 100 at another point during the method 800. As shown in FIG. 9, the catheter 1000 includes an elongate shaft 1002 and a first neuromodulation element 1004 operably connected to the shaft 1002. FIGS. 10 and 11 are partially cross-sectional profile views illustrating the sheath 900 and a catheter 1000 within the renal vasculature 100 at still other respective points during the method 800. FIG. 12 is an enlarged, partially cross-sectional profile view illustrating the sheath 900 and one embodiment of the catheter 1000 within the renal vasculature 100 at yet another point during the method 800.

As shown in FIG. 9, the catheter 1000 includes a first neuromodulation element 1004 operably connected to the shaft 1002. In one embodiment, the first neuromodulation element 1004 can include an elongate support structure 1008 carrying a plurality of longitudinally spaced-apart electrodes 1010 (e.g., between about 2 electrodes and about 8 electrodes, greater than 2 electrodes, etc.). As shown in FIG. 9, the elongate support structure 1008 can have a helical/spiral form when unconstrained. In some embodiments, for example, the catheter 1000 can be delivered over a guidewire (not shown) and the guidewire can be retracted to release a preformed helical/spiral configuration of the support structure 1008. In other embodiments, the sheath 900 can be a straining sheath or guide catheter and the treatment catheter 1000 can be deployed into the helical/spiral form when pushed or otherwise presented distally from the sheath 900. Once deployed, the plurality of longitudinally spaced-apart electrodes 1010 can be positioned in apposition with an inner wall of the vessel lumen for treatment (e.g., neuromodulation of nervous tissue proximal the inner wall). Examples of suitable multi-electrode devices are described in U.S. patent application Ser. No. 13/281,360, filed Oct. 25, 2011, and incorporated herein by reference in its entirety. Other suitable devices and technologies, are described in U.S. patent application Ser. No. 13/279,330, filed Oct. 23, 2011, and additional thermal devices are described in U.S. patent application Ser. No. 13/279,205, filed Oct. 21, 2011, each of which are incorporated herein by reference in their entireties.

As shown in FIG. 9, the helical/spiral form of the elongate support structure 1008 can be deployed in the distalmost portion of the main vessel 102 and/or in one or more of the branch vessels 110a, 110b, 114. The helical/spiral shape can be longitudinally compressed, longitudinally stretched/elongated and/or be capable of accommodating a variety of anatomically restricted or expanded vessel architectures. As such, neuromodulation using the catheter 100 can result in a compressed spiral lesion pattern (e.g., lesions radially spaced about 2 mm apart), an elongated spiral lesion pattern (e.g., lesions longitudinally spaced about 2 mm to about 5 mm apart) and/or some combination of both a compressed and elongated spiral lesion pattern at one or more anatomical locations along the inner wall of the renal vasculature.

In several embodiments, the first neuromodulation element 1004 can be deployed in a branch vessel 110a, 110b, 114 and neuromodulation using the first neuromodulation element 1004 can result in a spiral/helical lesion pattern within the branch vessel. In a particular embodiment, the spiral/helical lesion pattern can be positioned within a trunk segment of the branch vessel. For example, the lesions can be placed in a spiral/helical pattern or other pattern (e.g., zig-zag pattern) in a longitudinal segment of the branch vessel 110a, 110b, 114 spanning between about 0.5 mm to about 7 mm distal to the bifurcation, about 0.5 mm to about 6 mm, about 1 mm to about 6 mm, about 1 mm to about 5 mm, about 2 mm to about 5 mm, or in a further embodiment, spanning between about 2 mm to about 7 mm distal to the bifurcation. In additional examples, the lesions can be placed in a spiral/helical pattern or another pattern in a longitudinal segment of the one or more branch vessels 110a, 110b, 114 spanning between 0.5 mm to about 10 mm, about 1 mm to about 13 mm, about 1.5 mm to about 10 mm, about 2 mm to about 8 mm, about 3 mm to about 9 mm, or about 4 mm to about 12 mm distal to the primary bifurcation 108. In some embodiments, more than one branch vessel 110a, 110b, 114 can be treated. In a particular example, all accessible branch vessels can be treated. Following neuromodulation of the one or more branch vessels 110a, 110b, 114, the first neuromodulation element can be retracted proximally to a segment (e.g., the distalmost portion, the central portion, the proximal portion) of the main vessel 102 for optionally administering additional treatment. In various arrangements, the first neuromodulation element can be repositioned proximally from the branch vessel 110a, 110b, 114 to the main vessel 102 while fully deployed, partially compressed, or fully compressed into a low-profile delivery state before administering treatment to the main vessel 102.

Referring to FIG. 9, and in another embodiment, the catheter 1000 can optionally include a second neuromodulation element 1006 operably connected to the shaft 1002 and/or the first neuromodulation element 1004. The second neuromodulation element 1006 can include an elongate conduit 1011 directly connected to a distal end of the support structure 1008. For example, in the embodiment shown in FIG. 12, the second neuromodulation element 1006 further includes a wire electrode 1300. The support structure 1008 and the wire electrode 1300 can have respective helical/spiral forms when unconstrained. In another embodiment, the second neuromodulation element 1006 includes an elongate support structure similar to the support structure 1008 and includes electrodes similar to the electrodes 1010. Other variations of the first and second neuromodulation elements 1004, 1006 are also possible.

In FIG. 8, the first and second neuromodulation elements 1004, 1006 are in their respective low-profile delivery states within the sheath 900. The shaft 1002 can be advanced to the renal vasculature 100 with the first and second neuromodulation elements 1004, 1006 in these respective low-profile delivery states. For example, the sheath 900 and the conduit 1011 can constrain the support structure 1008 and the wire electrode 1300, respectively, into generally linear forms. As shown in FIG. 9, when the first neuromodulation element 1004 is at a desired position within the renal vasculature 100, constraint on the support structure 1008 can be reduced, such as by uncovering the support structure 1008 from within the sheath 900. This causes the support structure 1008 to assume its helical/spiral form, thereby moving the electrodes 1010 toward an inner surface of a wall of the main vessel 102.

As shown in FIG. 10, after delivering energy to the nerve fibers 124 within a distal portion of the first anatomical region 125a, the support structure 1008 is refracted relative to the sheath 900 and/or the sheath 900 advanced relative to the support structure 1008 to force the support structure 1008 back into its low-profile delivery state. The second neuromodulation element 1006 may remain protruding from a distal end of the sheath 900. As shown in FIG. 11, the second neuromodulation element 1006 can then be positioned within one of the branch vessels 110a, 110b, 114 while the wire electrode 1300 is in its low-profile delivery state. When the second neuromodulation element 1006 is properly positioned (as shown in FIG. 12), the second neuromodulation element 1006 is deployed into an expanded treatment state. For example, the wire electrode 1300 can be pushed distally out of the conduit 1011 (e.g., by pushing a control wire (not shown)) distally. This is expected to reduce constraint on the wire electrode 1300 such that the wire electrode 1300 assumes its helical/spiral form and contacts an inner surface of a wall of the branch vessel 110a, 110b, 114. In another example, the second neuromodulation element 1006 can have a single element tapering distally (e.g., a cross-sectional dimension of the second neuromodulation element 1006 may decrease from proximal to distal) to accommodate the inferior dimensions of the branch vessels 110a, 110b, 114. In these and other cases, the first and second neuromodulation elements 1004, 1006 can be continuous. For example, a distal portion of a single elongate support element can be deployed within one of the branch vessels 110a, 110b, 114 while a proximal portion of the same element is deployed within the distal portion of the main vessel 102. This can allow nerve fibers 124 on both sides of the primary bifurcation 108 to be treated simultaneously or consecutively, if desired.

Although FIGS. 7-12 illustrate neuromodulation of nerve tissue in the anatomical region 125a surrounding the main vessel 102 prior to neuromodulation of the nerve tissue in the anatomical regions 125b, 125c surrounding the branches 110a, 110b, 114, it should be understood that, in some embodiments, the anatomical regions 125b, 125c may be treated prior to the anatomical region 125a surrounding the main vessel 102.

With reference again to FIGS. 7-12 together, after reducing constraint on the wire electrode 1300, the method 800 includes using the second neuromodulation element 1006 to modulate nerve tissue within a portion of the second and/or third anatomical regions 125b, 125c extending circumferentially around the branch vessel 110a, 110b, 114 in which the second neuromodulation element 1006 is deployed. In FIG. 12, treatment of the second primary branch vessel 110b and two additional subordinate branch vessels 114 is shown in dashed lines. With respect to some embodiments, the dashed lines represent sequential treatment of these additional vessels. In another embodiment, the second neuromodulation element includes multiple wire electrodes 1300 that are individually guided into respective additional vessels. With respect to these embodiments, the dashed lines represent simultaneous treatment of the additional vessels.

Any one of the catheters described above with references to FIGS. 1-12 can be incorporated into a suitable neuromodulation system. FIG. 13, for example, is a partially schematic perspective view illustrating a neuromodulation system 1400 configured in accordance with one embodiment of the present technology. The system 1400 includes a console 1402, a catheter 1408, and a cable 1406 extending therebetween. The catheter 1408 can be identical or similar to any of the catheters described herein. The catheter 1408, for example, includes a handle 1404 and an elongate shaft 1410 having a proximal end portion 1410a and a distal end portion 1410b. The catheter 1408 further includes a neuromodulation element 1412 at the distal end portion 1410b of the shaft 1410. The shaft 1410 is configured to locate the neuromodulation element 1412 at a treatment location within a body lumen, such as a suitable blood vessel, duct, airway, or other naturally occurring lumen within the human body at any suitable branching level. Once located, the neuromodulation element 1412 is configured to provide or support a neuromodulation treatment.

The console 1402 is configured to control, monitor, supply energy to, and/or otherwise support operation of the catheter 1408. Alternatively, the catheter 1408 can be self-contained or otherwise configured for operation without connection to the console 1402. When present, the console 1402 can be configured to generate a selected form and/or magnitude of energy for delivery to tissue at a treatment location via the neuromodulation element 1412. The console 1402 can have different configurations depending on the treatment modality of the catheter 1408. For example, when the catheter 1408 is configured for electrode-based, heat-element-based, or transducer-based treatment, the console 1402 can include an energy generator (not shown) configured to generate radio frequency (RF) energy (e.g., monopolar and/or bipolar RF energy), pulsed electrical energy, microwave energy, optical energy, ultrasound energy (e.g., high-intensity focused ultrasound energy), direct heat, radiation (e.g., infrared, visible, and/or gamma radiation), and/or one or more other suitable types of energy.

The system 1400 can include a mechanical control device 1413 (e.g., a lever) configured to mechanically control operation of one or more components of the catheter 1408. The system 1400 can further include an electrical control device 1414 configured to electrically control operation of one or more components of the catheter 1408 directly and/or via the console 1402. The electrical control device 1414 can be disposed along the cable 1406 as shown in FIG. 13, incorporated into the handle 1404, or have another suitable position within the system 1400. During use of the system 1400, an operator can use the electrical control device 1414 to provide instructions to the console 1402, such as to initiate or terminate a neuromodulation treatment. In addition to being configured to execute such instructions, the console 1402 can be configured to execute an automated control algorithm 1416. Furthermore, the console 1402 can be configured to provide information to an operator before, during, and/or after a neuromodulation procedure via a feedback algorithm 1418. Feedback from the feedback algorithm 1418 can be audible, visual, haptic, or have another suitable form. The feedback can be based on output from a monitoring system (not shown). For example, such a monitoring system can include a monitoring device (e.g., a sensor) configured to measure a condition at a treatment location (e.g., a temperature of tissue being treated), a systemic condition (e.g., a patient vital sign), or another condition germane to the treatment, health, and/or safety of a patient. The monitoring device can be integrated into the catheter 1408 or separate from the catheter 1408.

### Renal Neuromodulation

Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along nerve fibers (e.g., efferent and/or afferent nerve fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions. Such clinical conditions may be the result of physiological parameters associated with systemic sympathetic overactivity or hyperactivity such as elevated blood pressure, elevated blood sugar levels, ovarian cysts, etc.

Renal neuromodulation can be electrically-induced, thermally-induced, or induced in another suitable manner or combination of manners at one or more suitable treatment locations during a neuromodulation procedure. The treatment location can be within or otherwise proximate to renal vasculature (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a neuromodulation procedure can include modulating nerves in the renal plexus, which lay intimately within or adjacent to the adventitia of the renal artery. Various suitable modifications can be made to the catheters described above to accommodate different treatment modalities. For example, the electrodes 1010 (FIG. 9) can be replaced with transducers to facilitate transducer-based treatment modalities.

Renal neuromodulation can include an electrode-based or treatment modality alone or in combination with another treatment modality. Electrode-based or transducer-based treatment can include delivering electricity and/or another form of energy to tissue at or near a treatment location to stimulate and/or heat the tissue in a manner that modulates nerve function. For example, sufficiently stimulating and/or heating at least a portion of a sympathetic renal nerve can slow or potentially block conduction of neural signals to produce a prolonged or permanent reduction in renal sympathetic activity (e.g., reducing sympathetic neural activity). A variety of suitable types of energy can be used to stimulate and/or heat tissue at or near a treatment location. For example, neuromodulation in accordance with embodiments of the present technology can include delivering RF energy, pulsed electrical energy, microwave energy, optical energy, focused ultrasound energy (e.g., high-intensity focused ultrasound energy), and/or another suitable type of energy. An electrode or transducer used to deliver this energy can be used alone or with other electrodes or transducers in a multi-electrode or multi-transducer array.

Neuromodulation using focused ultrasound energy (e.g., high-intensity focused ultrasound energy) can be beneficial relative to neuromodulation using other treatment modalities. Focused ultrasound is an example of a transducer-based treatment modality that can be delivered from outside the body. Focused ultrasound treatment can be performed in close association with imaging (e.g., magnetic resonance, computed tomography, fluoroscopy, ultrasound (e.g., intravascular or intraluminal), optical coherence tomography, or another suitable imaging modality). For example, imaging can be used to identify an anatomical position of a treatment location (e.g., as a set of coordinates relative to a reference point). The coordinates can then entered into a focused ultrasound device configured to change the power, angle, phase, or other suitable parameters to generate an ultrasound focal zone at the location corresponding to the coordinates. The focal zone can be small enough to localize therapeutically-effective heating at the treatment location while partially or fully avoiding potentially harmful disruption of nearby structures. To generate the focal zone, the ultrasound device can be configured to pass ultrasound energy through a lens, and/or the ultrasound energy can be generated by a curved transducer or by multiple transducers in a phased array, which can be curved or straight.

Heating effects of electrode-based or transducer-based treatment can include ablation and/or non-ablative alteration or damage (e.g., via sustained heating and/or resistive heating). For example, a neuromodulation procedure can include raising the temperature of target nerve fibers to a target temperature above a first threshold to achieve non-ablative alteration, or above a second, higher threshold to achieve ablation. The target temperature can be higher than about body temperature (e.g., about 37° C.) but less than about 45° C. for non-ablative alteration, and the target temperature can be higher than about 43° C. for ablation. Heating tissue to a temperature between about body temperature and about 43° C. can induce non-ablative alteration with reversible consequences, for example, via moderate heating of target nerve fibers or of luminal structures that perfuse the target nerve fibers. In cases where luminal structures are affected, the target nerve fibers can be denied perfusion resulting in necrosis of the nerve tissue. Heating tissue to a target temperature higher than about 43° C. (e.g., higher than about 60° C.) can induce ablation, for example, via substantial heating of target nerve fibers or of luminal structures that perfuse the target fibers. In some patients, it can be desirable to heat tissue to temperatures that are sufficient to ablate the target nerve fibers or the luminal structures, but that are less than about 90° C. (e.g., less than about 85° C., less than about 80° C., or less than about 75° C.).

### EXPERIMENTAL EXAMPLES

### Example 1

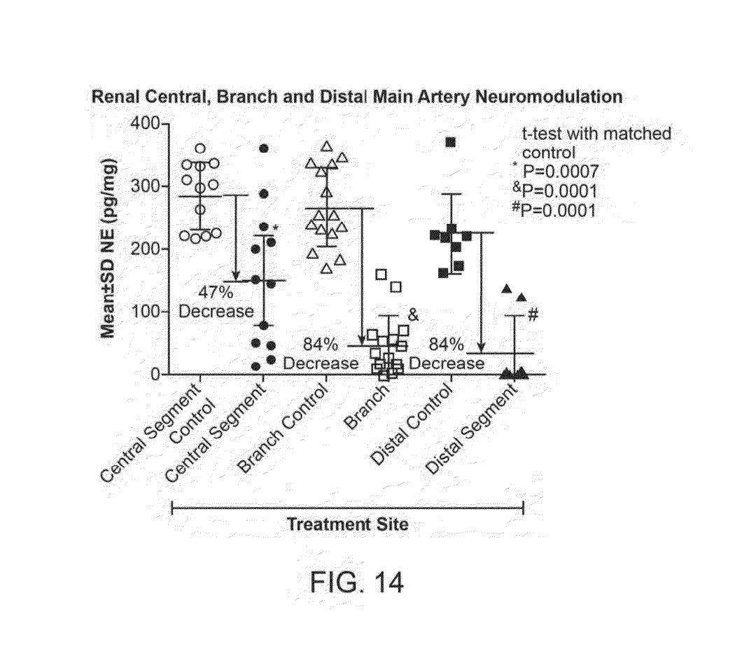

This section describes an example of the outcome of renal neuromodulation on animal subjects. In this example, and referring to FIG. 14, studies using the pig model have been performed directed to modulation of nerve tissue at different treatment sites within the renal vasculatures using a multi-electrode Symplicity Spyral™ catheter or a single-electrode Symplicity Flex™ catheter, both commercially available from Medtronic, Inc., of Minneapolis, Minn. Renal cortical norepinephrine (NE) concentration was used as a surrogate marker to measure procedural efficacy.

FIG. 14 is a plot of mean NE concentration before and after renal neuromodulation procedures performed at the central segment (e.g., “Central”) of the main renal artery, the distal segment (e.g., “Distal”) of the main renal artery, and in two renal artery branches (e.g., “Branch”) of pig subjects. For pigs undergoing treatment of a branch vessel site, a Symplicity Spyral™ catheter was inserted entirely into the cranial renal artery branch and used to treat the branch with four lesions at the furthest distance into the branch vessel that could accommodate the device given dimensional constraints. The catheter was then withdrawn into the main renal vessel and then advanced under fluoroscopy into the caudal renal artery branch and treatment procedure was repeated. In all cases with three or more renal artery branches only the cranial and caudal branch were treated. Accordingly, the multi-electrode device was advanced so that the electrodes were inserted as far as possible into the branches. For pigs undergoing distal main renal artery treatment, six lesions were formed at the distal segment of the renal artery and within a distance of 6 mm proximal to the branch point within the renal artery using the Symplicity Flex™ catheter (FIG. 14, “Distal Portion”). The longitudinal spacing between the lesions was approximately 2 mm, with a lesion footprint of approximately 2 mm each. In this experimental example, a first lesion was formed about 5-6 mm from the bifurcation. The catheter was then proximally retracted 1-2 mm (e.g., maximum of 2 mm) and rotated 90 degrees followed by formation of a second lesion. Further lesions were formed by sequential movement of the catheter proximally 1-2 mm, rotation of 90 degrees followed by lesion formation. As such, a longitudinal separation of lesions occurred approximately 1-2 mm apart along the longitudinal length of the distal segment of the main renal artery. For pigs undergoing main artery treatment at a central segment of the main renal artery, a Symplicity Flex™ catheter was used to form between 4 and 6 ablations in a spiral/helical pattern along the central segment of the main renal artery. In these animals, the first lesion was placed approximately 5 mm proximal to the bifurcation. Each subsequent lesions was placed 5 mm proximally and rotated at 90 degrees forming a spiral pattern.

As shown in FIG. 14, mean renal cortical NE levels dropped approximately 84% between control and experimental groups for pigs undergoing treatment at the distal segment of the main renal artery or at a branch artery. Pigs undergoing treatment at the central segment of the main renal artery exhibited an approximate 47% decrease in mean NE levels following treatment. Accordingly, the distal segment and branch treatment sites exhibited an increased effectiveness of neuromodulation as compared to treatment at the central segment as indicated by NE concentration in renal tissue (FIG. 14).

These findings suggest that the positioning of the treatment device within the renal vasculature at a branch vessel or a distal portion of the main renal artery, as measured by NE concentration in renal tissue, results in increased efficacy of modulation of targeted renal nerves. These findings also suggest that the position in the distal segment of the main renal artery (e.g., distalmost third of the main renal vessel, distalmost quarter of the main renal vessel, approximately 1 cm proximal of the branch point to approximately 6 cm proximal of the branch point, between approximately 1 cm and approximately 10 cm proximal of the main renal vessel bifurcation, etc.) can provide a target for RF nerve ablation due to the proximity of the renal nerves. For example, in some embodiments, a compressed spiral/helical lesion pattern in a segment of the renal artery wherein the renal nerves are consistently in closer proximity to the inner wall of the renal vessel can effectively treat more nerves than a set of helical lesions spaced further apart along the full length of the main renal artery where distribution of the renal nerves vary in number, orientation and proximity to the to the inner wall of the renal vessel.

### Example 2

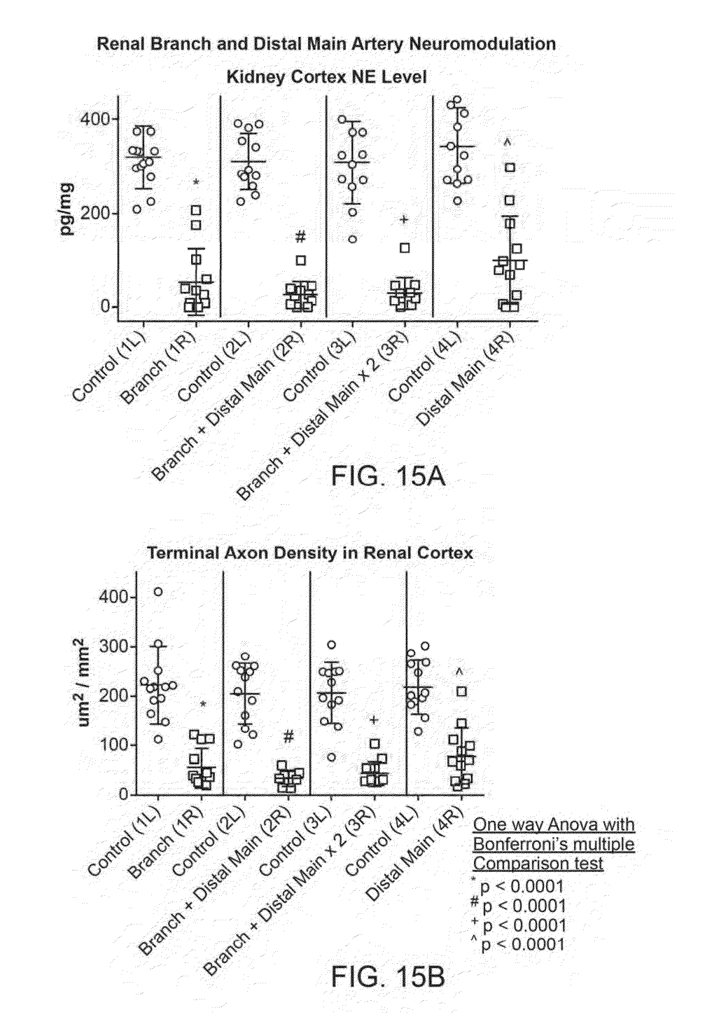

Example 2 also describes of the outcome of renal neuromodulation on animal subjects in an additional experiment. In this example, and referring to FIGS. 15A and 15B, studies using the pig model have been performed directed to modulation of nerve tissue at different treatment sites and/or different combinations of treatment sites within the renal vasculature using a multi-electrode Symplicity Spyral™ catheter or a single-electrode Symplicity Flex™ catheter along with a Symplicity G3™ generator, all of which are commercially available from Medtronic, Inc., of Minneapolis, Minn. Both renal cortical NE concentration (FIG. 15A) and terminal axon density in the renal cortex (FIG. 15B) were used as markers to measure procedural efficacy.

FIG. 15A is a plot of mean NE concentration before and after renal neuromodulation procedures performed at the distal segment (e.g., distal third) of the main renal artery (e.g., “Distal Main”), in two renal artery branches (e.g., “Branch”), at the renal artery branches and the main renal artery (e.g., “Branch+Main”), and at the renal artery branches and two cycles of treatment in the main renal artery (e.g., “Branch+Main×2”) of pig subjects (N=12 pigs). For pigs undergoing treatment of a branch vessel site, a Symplicity Spyral™ catheter was inserted into the first renal artery branch (e.g., the cranial branch) and used to treat the branch with up to four lesions (e.g., 2 lesions, 3 lesions, 4 lesions) with distalmost electrodes being optionally and selectively deactivated in some instances in which the branch vessel was short, narrow, or otherwise undesirable for treatment. The catheter was placed in the first branch location such that the proximal-most electrode was distal to the bifurcation and, when deployed, the lesions were approximately 2 mm apart. The catheter was then withdrawn into the main renal vessel and then advanced under fluoroscopy into the second renal artery branch (e.g., the caudal branch) and the treatment procedure was repeated. In all cases with three or more renal artery branches only the cranial and caudal branch were treated. Accordingly, the multi-electrode device was advanced so that the first electrode (e.g., proximal electrode) was placed approximately 1-3 mm distal to the bifurcation.

For pigs undergoing distal main renal artery treatment, six lesions were formed at the distal segment of the renal artery and within a distance of 6 mm proximal to the branch point within the renal artery using the Symplicity Flex™ catheter (FIGS. 15A-15B, “Distal Main”). The longitudinal spacing between the lesions was approximately 2 mm, with a lesion footprint of approximately 2 mm each. In this experimental example, a first lesion was formed about 5-6 mm proximal to the bifurcation. The catheter was then proximally retracted 1-2 mm (e.g., maximum of 2 mm) and rotated 90 degrees followed by formation of a second lesion. Further lesions were formed by sequential movement of the catheter proximally 1-2 mm, rotation of 90 degrees followed by lesion formation. As such, a longitudinal separation of lesions occurred approximately 1-2 mm apart along the longitudinal length of the distal segment of the main renal artery.

In pigs undergoing a combination treatment approach that includes both treatment of the branches and the distal portion of the main renal artery, a Symplicity Spyral™ catheter was inserted into the first renal artery branch for treatment, followed by the second renal artery branch for treatment, and finally retracted and deployed for treatment in the distal portion of the main renal artery.

As shown in FIG. 15A, mean renal cortical NE levels dropped approximately greater than 80% between control and experimental groups for pigs undergoing treatment at the distal segment of the main renal artery (e.g., “Distal Main”) or at the branch arteries (e.g., “Branch”). Neuromodulation in these pigs also resulted in approximately 70% reduction (“Distal Main”) and greater than 80% reduction (“Branch”) in cortical axonal density (FIG. 15B). In this example, untreated or undertreated segments (e.g., branches treated with only 2 or 3 lesions) can give rise to outliers in the data set (e.g., note two outliers approaching 200 pg/mg NE in the treated branch column “Branch (1R)” of FIG. 15A). Pigs undergoing a combination treatment approach that combined treatment of the branches and the distal portion of the main renal artery using a Symplicity Spyral™ catheter demonstrated a greater than 90% reduction in mean renal NE concentration. The 90% reduction in NE was corroborated with a 92% reduction terminal axon density as shown in FIG. 15B. Pigs undergoing another combination treatment approach that combined treatment of the branches and two treatment cycles of the distal portion of the main renal artery using a Symplicity Spyral™ catheter also exhibited a greater than 90% reduction in mean renal NE concentration levels and a 90% reduction in terminal axon density following treatment (FIGS. 15A and 15B, respectively), but did not statistically improve the response more than the combination treatment approach that included a single cycle of treatment in the main renal artery. Accordingly, the branch treatment sites combined with treatment of the distal portion of the main renal artery exhibited an increased effectiveness of neuromodulation as compared to treatment at the distal segment or the branches alone as indicated by NE concentration levels and terminal axon density in renal tissue (FIGS. 15A and 15B).

These findings suggest that the positioning of the treatment device within the renal vasculature at a branch vessel for treatment followed by treatment of the main renal artery, as measured by NE concentration in renal tissue and histologically by measuring terminal axon density in the renal cortex, is expected to result in increased efficacy of modulation of targeted renal nerves. These findings also suggest that a combination approach of treating the branches (e.g., at least two branches) in addition to one or more segments of the main renal artery (e.g., the proximal portion, the central portion, the distalmost third of the main renal vessel, distalmost quarter of the main renal vessel, approximately 1 cm proximal of the branch point to approximately 6 cm proximal of the branch point, between approximately 1 cm and approximately 10 cm proximal of the main renal vessel bifurcation, etc.), can provide a targeted therapeutic approach for RF nerve ablation. Without being bound by theory, the targeted therapeutic approach for RF nerve ablation is expected to have higher efficacy in the combined treatment approach in certain instances due to the proximity of the renal nerves to the renal vessel wall in the distal segment of the main renal artery (e.g., distalmost third of the main renal vessel, distalmost quarter of the main renal vessel, approximately 1 cm proximal of the branch point to approximately 6 cm proximal of the branch point, between approximately 1 cm and approximately 10 cm proximal of the main renal vessel bifurcation, etc.) and in the branches. Additionally, by combining the treatment locations (e.g., branches and main renal artery) into a treatment session, more renal nerves may be ablated.

### Example 3

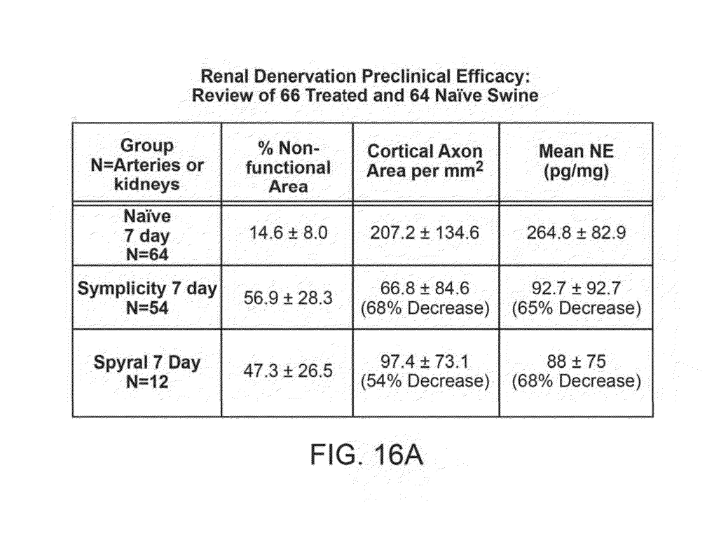

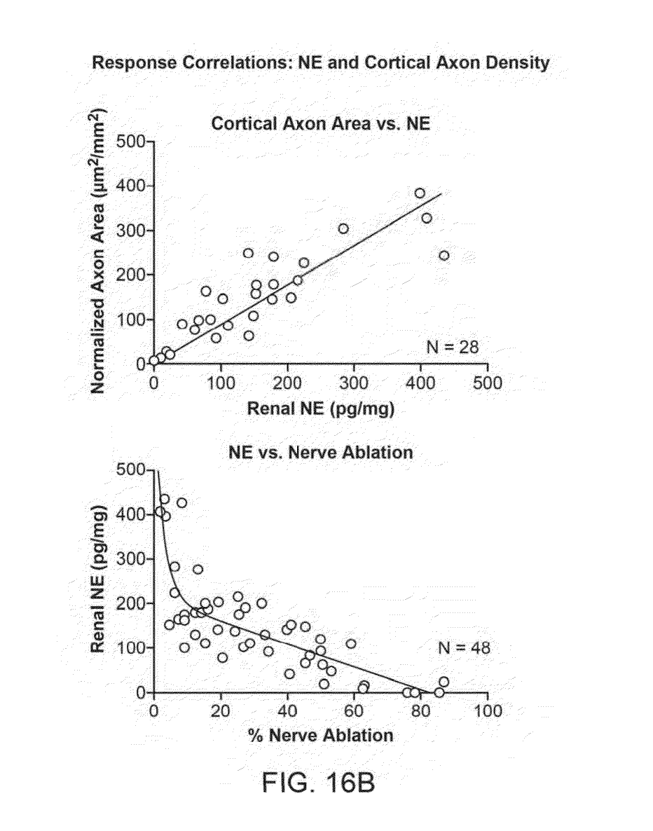

Example 3 describes the outcome of catheter-based renal neuromodulation on animal subjects in an additional experiment. In this example (and referring to FIGS. 16A and 16B), studies using the pig model were performed using a multi-electrode Symplicity Spyral™ catheter or a single-electrode Symplicity Flex™ catheter along with a Symplicity G3™ generator. The catheters and generator are commercially available from Medtronic, Inc., of Minneapolis, Minn. The catheters were used in these cohorts of animals (n=66) to create multiple RF ablations in the renal vasculature. Cortical axon density in the renal cortex (FIG. 16A) and renal cortical NE concentration (FIG. 16B) were used as markers to measure procedural efficacy.

As shown in FIG. 16A, cortical axon area (per mm²) dropped approximately greater than 54% between a control group (n=64) and treated groups of pigs (n=66) undergoing treatment. For pigs undergoing treatment with the Symplicity Flex™ catheter (n=54), an average of 4.1 lesions were formed in each animal. These pigs demonstrated a 56.9% increase in non-functional axonal area along the renal artery, and a 68% decrease in cortical axon area as compared with the control group. For pigs undergoing treatment with the Symplicity Spyral™ catheter (n=12), an average of 4.0 lesions were formed in each animal. The pigs undergoing treatment with the Symplicity Spyral™ catheter demonstrated a 47.3% increase in non-functional area along the renal artery, and a 54% decrease in cortical axon area relative to the control group. Without being bound by theory, it is believed that the loss of cortical axons is a likely consequence of nerve atrophy distal to the ablation sites.

FIG. 16B includes (a) a graph of normalized cortical axon area vs. renal NE concentration, and (b) a graph of renal NE concentration vs. extent (%) of nerve ablation. Referring to the table of FIG. 16A and the two graphs of FIG. 16B together, cortical axon area correlates directly with renal NE. In particular, pigs undergoing treatment with the Symplicity Flex™ catheter exhibited a 65% decrease in mean NE level compared with the pigs in the control group. The pigs treated with the Symplicity Spyral™ catheter exhibited a 68% decrease in mean NE level compared with the pigs in the control group. This is further shown by the first graph of FIG. 16B, which demonstrates that a decrease in cortical axon area correlates with a decrease in NE levels. Referring to the second graph of FIG. 16B, renal NE decrease is non-linear with increased loss of nerve viability along the artery (further extent (%) of nerve ablation). These findings suggest that catheter-based renal neuromodulation exhibits a relatively consistent impact on sympathetic nerve function and viability.

### Example 4

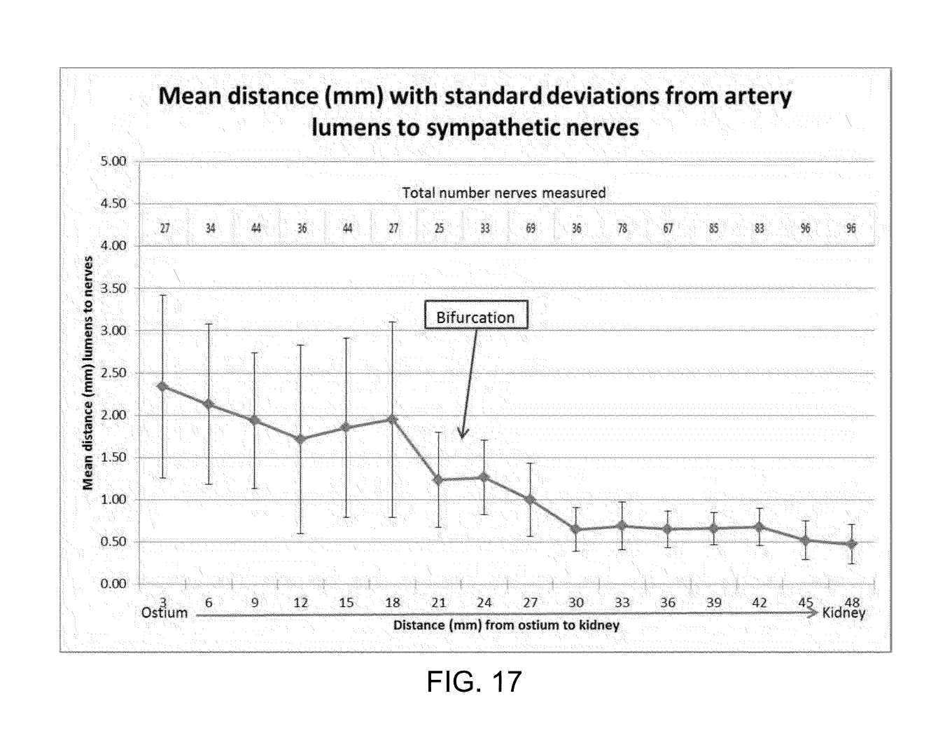

Example 4 describes the results of mapping the location of sympathetic nerves relative to an artery lumen along the renal vasculature from a renal ostium to a kidney of a representative human patient. FIG. 17, for example, is a line graph illustrating the mean distance (mm) with standard deviation between an arterial lumen and sympathetic nerves in proximity to and extending along the length of the renal vasculature. In this example, cross-sections of renal tissue along the renal vasculature were collected and stained for sympathetic nerve tissue and other anatomical features (not shown). Histology tissue slices were collected every 3 mm from between the renal ostium and the kidney. Histological images were analyzed and measurement between the arterial lumen and the sympathetic nerves in proximity to the lumens were captured utilizing Aperio® ImageScope software (available from Leica Biosystems Inc. of Buffalo Grove, Ill.).

Referring to FIG. 17, mean distance (mm) between the sympathetic nerves measured (total number for each position along vasculature recorded along the top of the graph) and the lumen of the main renal artery, bifurcation and branch artery are represented by the line graph. As shown in FIG. 17, the mean distance between sympathetic renal nerves and the arterial lumens generally decreases along the main renal artery as progressing from the ostium to the kidney. For example, at the ostium (e.g., proximal portion of the main renal artery), the mean distance of sympathetic nerves (n=27) from the artery lumen is about 2.4 mm; at the central/middle portion of the main renal artery, the mean distance is about 1.75 mm (n=36); and at the distal portion of the main renal artery, the mean distance is about 1.25 mm (n=25).

The findings also show that the mean distance between the sympathetic nerves and the arterial lumen continues to decrease in positions distal to the bifurcation of the renal artery (shown with arrow; FIG. 17). For example, at 24 mm distal to the ostium (e.g., 1.5 mm distal to the bifurcation), the mean distance of sympathetic nerves (N=33) from the branch lumen is about 1.25 mm; at 27 mm distal to the ostium (e.g., 4.5 mm distal to the bifurcation), the mean distance is about 1.0 mm (n=69); and at 30 mm distal to the ostium (e.g., 7.5 mm distal to the bifurcation), the mean distance is about 0.65 mm (n=36). These findings also show that deviation from the calculated mean distance of individual nerves have less variance at locations more distal to the bifurcation as compared to other locations along the arterial structure. As shown in FIG. 17, for example, the mean distance between the sympathetic nerves and the arterial branch lumen remains relatively constant in regions greater than about 7.5 mm distal to the bifurcation.

As further shown in FIG. 17, the total number of sympathetic nerves proximal (close to) to the arterial lumens remains relatively constant along the main renal artery and the bifurcation. However, the total number of sympathetic nerves proximal (close to) to the arterial lumen significantly increases in the region distal to the bifurcation. Accordingly, the data shown in FIG. 17 suggests that the proximity of nerve fibers along portions of the renal vasculature increases in a proximal to distal direction (e.g., the nerve fibers are closer to the arterial structure in branch regions compared to the main renal artery). Further, the data shown in FIG. 17 suggests that additional nerve fibers approach the wall of the arterial vasculature in branch regions distal to the primary bifurcation.

These findings suggest that the positioning of a treatment device within the renal vasculature at a branch vessel for treatment, as indicated by the increased proximity of target nerve fibers to the renal vasculature in this region and by the increased number of target nerve fibers in this region, is expected to result in increased efficacy of modulation of targeted renal nerves. These findings also suggest that a targeted therapeutic approach for renal neuromodulation (e.g., RF nerve ablation) in regions distal to approximately 1.5 mm of the bifurcation, between approximately 1.5 mm and approximately 10 mm or more distal to the bifurcation, between approximately 2 mm and approximately 8 mm distal to the bifurcation, between approximately 3 mm and approximately 9 mm distal to the bifurcation, between approximately 4 mm and approximately 12 mm distal to the bifurcation, or between approximately 1 mm and approximately 7 mm distal to the bifurcation, can result in higher efficacy. In this particular human subject, the length of the arterial vasculature between the ostium and the kidney is approximately 48 mm with approximately 24 mm between the primary bifurcation and the kidney. In certain embodiments, a targeted therapeutic approach for renal denervation in a particular patient can include at least one treatment site in region(s) of the branch vessels. Without being bound by theory, by targeting such treatment sites in a treatment session, more renal nerves may be ablated given their proximity to the vessel. Further, by combining treatment locations (e.g., branches and a distal portion of the main renal artery) into a single treatment session, it is expected that a greater volume of renal nerves may be ablated.

### Example 5

Example 5 describes a method for treating human patients with renal denervation and anticipated outcomes of such treatment. In this example, human patients will be treated with renal denervation and a method of treatment includes modulating nerve tissue surrounding one or more primary branch trunks (e.g., proximal portion of one or more primary branch vessels distal to the bifurcation). In this example, modulating nerve tissue includes forming up to about four lesions (e.g., about 2 lesions to about 4 lesions) in the primary branch trunk from about 1 mm to about 5 mm distal to the primary bifurcation or, in another example, from about 2 mm to about 6 mm distal to the primary bifurcation. In additional examples, the lesions can be formed through the wall of the branch vessel within the longitudinal length spanning about 0.5 mm to about 10 mm, about 1 mm to about 13 mm, about 1.5 mm to about 10 mm, about 2 mm to about 8 mm, about 3 mm to about 9 mm, about 4 mm to about 12 mm, or about 1 mm to about 7 mm, distal to the primary bifurcation. Modulation of nerve tissue at branch trunk treatment sites and/or different combinations of treatment sites within the renal vasculature can be performed using a single-electrode Symplicity Flex™ catheter or a multi-electrode Symplicity Spyral™ catheter, both commercially available from Medtronic, Inc. Other multi-electrode, spiral/helical-shaped catheters having a tighter spiral/helix (e.g., smaller pitch) for forming multiple lesions close in proximity along the length of the vessel are contemplated for these methods. Physiological biomarkers, such as systemic renin and aldosterone, or systemic catecholamines and/or their subsequent degradation products could be measured in either plasma, serum or urine to serve as surrogate markers to measure procedural efficacy such as described in International Patent Application No. PCT/US15/47568, filed Aug. 28, 2015, and incorporated herein by reference in its entirety.

A method for efficaciously neuromodulating renal nerve tissue in a human patient can include advancing a single-electrode Symplicity Flex™ catheter to a first renal artery branch vessel approximately 6 mm distal to the bifurcation. A first lesion can be formed about 5-6 mm distal to the bifurcation. The catheter can then be proximally retracted 1-2 mm (e.g., maximum of 2 mm) and rotated 90 degrees followed by formation of a second lesion. Further lesions can be formed by sequential movement of the catheter proximally 1-2 mm, rotation of 90 degrees followed by lesion formation. As such, a longitudinal separation of lesions can occur approximately 1-2 mm apart along the longitudinal length of the first renal artery branch vessel (e.g., first branch trunk). In other examples, the catheter can be rotated (e.g., 90 degrees) following formation of the first lesion such that discrete lesions (e.g., non-continuous) are formed in the same longitudinal plane. Following treatment at the first renal artery branch, the catheter can be withdrawn into the main renal vessel and then advanced under fluoroscopy into a second renal artery branch and the treatment procedure can be repeated. Some methods can include treating two branch vessels at the proximal trunk segment of the branch vessel. Other methods can include treating greater than two or all of the primary branch vessels branching from the main renal vessel (e.g., distal to a primary bifurcation). As described above, these methods may also include combining neuromodulation of renal nerve tissue surrounding one or more primary branch trunks with neuromodulation of renal nerve tissue at additional treatment location (e.g., locations along the main renal vessel, locations at or near the bifurcation, etc.). Other methods can include advancing a single-electrode Symplicity Flex™ catheter to a first renal artery branch vessel approximately 10 mm distal to the bifurcation. The first lesion can be formed about 9-10 mm distal to the bifurcation, and the catheter can then be proximally retracted and rotated for forming subsequent lesions as discussed above.

It is anticipated that the positioning of the treatment device within the renal vasculature at a branch vessel and forming lesions in a spiral/helical-shaped or near spiral/helical-shaped pattern within the trunk segment (e.g., proximal portion of one or more primary branch vessels distal to the bifurcation, from about 1-5 mm distal to the primary bifurcation, from about 2-6 mm distal to the primary bifurcation, etc.) or within another distal segment of the branch (e.g., from about 4-12 mm distal to the primary bifurcation, etc.) will result in increased efficacy of modulation of targeted nerves, as measured by levels of physiological biomarkers, such as systemic renin, aldosterone, or systemic catecholamines and degradation products thereof in plasma, serum or urine pre- and post-procedure (described in International Patent Application No. PCT/US15/47568, filed Aug. 28, 2015).

### Additional Examples

1. A method, comprising:
intravascularly advancing an elongate shaft of a catheter to renal vasculature of a human patient, the renal vasculature including—
   a main vessel directly connected to an aorta of the patient and extending distally toward a kidney, and
   a bifurcation at a distal end of the main vessel;
locating a neuromodulation element of the catheter within a distalmost portion of the main vessel; and
modulating nerve tissue within an anatomical region extending circumferentially around the distalmost portion of the main vessel via the neuromodulation element.

2. The method of example 1 wherein:
the main vessel has a longitudinal axis extending from the aorta to the bifurcation;
modulating nerve tissue within the anatomical region extending circumferentially around the distalmost portion of the main vessel includes using the neuromodulation element to form one or more lesions extending through a wall of the main vessel into the anatomical region extending circumferentially around the distalmost portion of the main vessel; and
the one or more lesions collectively are—
   circumferentially continuous within the anatomical region along a plane perpendicular to a portion of the longitudinal axis extending through the distalmost portion of the main vessel, and
   circumferentially discontinuous at the wall of the main vessel along all planes perpendicular to the portion of the longitudinal axis extending through the distalmost portion of the main vessel.

3. The method of example 1 or example 2 wherein the main vessel is stented, and wherein locating a neuromodulation element of the catheter within a distalmost portion of the main vessel includes locating the neuromodulation element distal to a stent.

4. The method of example 1 or example 2 wherein modulating nerve tissue within the anatomical region extending circumferentially around the distalmost portion of the main vessel includes using the neuromodulation element to preferentially modulate nerve tissue within the anatomical region extending circumferentially around the distalmost portion of the main vessel relative to nerve tissue within an anatomical region extending circumferentially around a proximal-most portion of the main vessel and relative to nerve tissue within an anatomical region extending circumferentially around a middle portion of the main vessel between the proximal-most and distalmost portions of the main vessel.

5. The method of any one of examples 1-4 wherein:
the catheter, the shaft, and the neuromodulation element are a first catheter, a first shaft, and a first neuromodulation element, respectively; and
the method further comprises—
   withdrawing the first catheter from the patient,
   intravascularly advancing an elongate second shaft of a second catheter to the renal vasculature,
   locating a second neuromodulation element of the second catheter within a branch vessel of the renal vasculature distal to the bifurcation, and
   modulating nerve tissue within an anatomical region extending circumferentially around the branch vessel via the second neuromodulation element.

6. The method of example 5, further comprising measuring a degree of neuromodulation achieved using the first neuromodulation element to modulate nerve tissue within the anatomical region extending circumferentially around the distalmost portion of the main vessel, and wherein locating the second neuromodulation element and modulating nerve tissue within the anatomical region extending circumferentially around the branch vessel includes locating the second neuromodulation element and using the second neuromodulation element to modulate nerve tissue within the anatomical region extending circumferentially around the branch vessel in response to an insufficiency of the degree of neuromodulation.

7. The method of any one of examples 1-4 wherein:
advancing the shaft includes advancing the shaft while the neuromodulation element is in a low-profile delivery state; and
the method further comprises transforming the neuromodulation element between the low-profile delivery state and an expanded treatment state after locating the neuromodulation element within the distalmost portion of the main vessel and before modulating nerve tissue within the anatomical region extending circumferentially around the distalmost portion of the main vessel.

8. The method of example 7 wherein:
the neuromodulation element includes a balloon; and
transforming the neuromodulation element includes inflating the balloon.

9. The method of example 7 wherein:
the neuromodulation element includes an elongate support structure carrying a plurality of electrodes, the support structure having a helical form when unconstrained;
advancing the shaft includes advancing the shaft while the support structure is constrained; and
transforming the neuromodulation element includes reducing constraint on the support structure such that the support structure moves toward having the helical form.

10. The method of example 7 wherein:
the neuromodulation element includes an elongate electrode having a helical form when unconstrained;
advancing the shaft includes advancing the shaft while the electrode is constrained; and
transforming the neuromodulation element includes reducing constraint on the electrode such that the electrode moves toward having the helical form.

11. The method of any one of examples 1-4 wherein:
the neuromodulation element is a first neuromodulation element; and
the method further comprises—
   locating a second neuromodulation element of the catheter within a branch vessel of the renal vasculature distal to the bifurcation, and
   modulating nerve tissue within an anatomical region extending circumferentially around the branch vessel after locating the second neuromodulation element.

12. The method of example 11 wherein:
advancing the shaft includes advancing the shaft while the second neuromodulation element is in a low-profile delivery state; and
the method further comprises transforming the second neuromodulation element between the low-profile delivery state and an expanded treatment state after locating the second neuromodulation element and modulating nerve tissue within the anatomical region extending circumferentially around the branch vessel.

13. The method of example 11 wherein:
the second neuromodulation element includes a balloon; and
transforming the second neuromodulation element includes inflating the balloon.

14. The method of example 11 wherein:
the second neuromodulation element includes an elongate support structure carrying a plurality of electrodes, the support structure having a helical form when unconstrained;
advancing the shaft includes advancing the shaft while the support structure is constrained; and
transforming the second neuromodulation element includes reducing constraint on the support structure such that the support structure moves toward having the helical form.

15. The method of example 11 wherein:
the second neuromodulation element includes an elongate electrode having a helical form when unconstrained;
advancing the shaft includes advancing the shaft while the electrode is constrained; and
deploying the second neuromodulation element includes reducing constraint on the electrode such that the electrode moves toward having the helical form.

16. The method of any one of examples 11-15, further comprising measuring a degree of neuromodulation achieved using the first neuromodulation element to modulate nerve tissue within the anatomical region extending circumferentially around the distalmost portion of the main vessel, wherein locating the second neuromodulation element and modulating nerve tissue within the anatomical region extending circumferentially around the branch vessel includes locating the second neuromodulation element and modulating nerve tissue within the anatomical region extending circumferentially around the branch vessel in response to an insufficiency of the degree of neuromodulation.

17. A method, comprising:
intravascularly advancing an elongate shaft of a catheter to renal vasculature of a human patient, the renal vasculature including—
   a main vessel directly connected to an aorta of the patient and extending distally toward a kidney,
   a bifurcation at a distal end of the main vessel, and
   a branch vessel distal to the bifurcation;
modulating nerve tissue within an anatomical region extending circumferentially around the main vessel;
measuring a degree of neuromodulation achieved by modulating nerve tissue within the anatomical region extending circumferentially around the main vessel; and
in response to an insufficiency of the degree of neuromodulation, modulating nerve tissue within an anatomical region extending circumferentially around the branch vessel.

18. The method of example 17 wherein the main vessel is stented.

19. The method of example 17 or example 18 wherein:
the degree of neuromodulation is a first degree of neuromodulation;
the branch vessel is a first branch vessel;
the renal vasculature includes a second branch vessel, the first and second branch vessels being independently connected to the main vessel; and
the method further comprises—
   measuring a second degree of neuromodulation after modulating nerve tissue within the anatomical region extending circumferentially around the first branch vessel; and
   in response to an insufficiency of the second degree of neuromodulation, modulating nerve tissue within an anatomical region extending circumferentially around the second branch vessel.

20. The method of example 19 wherein:
the renal vasculature includes a third branch vessel, the first, second, and third branch vessels being independently connected to the main vessel; and
the method further comprises—
   measuring a third degree of neuromodulation after modulating nerve tissue within the anatomical region extending circumferentially around the second branch vessel; and
   in response to an insufficiency of the third degree of neuromodulation, modulating nerve tissue within an anatomical region extending circumferentially around the third branch vessel.

21. A method including any non-conflicting combination of the preceding examples 1-20.

22. A method, comprising:
intravascularly positioning a neuromodulation element of a catheter within renal vasculature of a human patient, the renal vasculature including—
   a main vessel directly connected to an aorta of the patient and extending distally toward a kidney,
   a bifurcation at a distal end of the main vessel, and
   a branch vessel distal to the bifurcation;
modulating nerve tissue within an anatomical region extending circumferentially around the branch vessel; and modulating nerve tissue within an anatomical region extending circumferentially around the main vessel.

23. The method of example 22 wherein the branch vessel is a first branch vessel, and wherein the method further comprises modulating nerve tissue within an anatomical region extending circumferentially around a second branch vessel, the second branch vessel distal to the bifurcation.

24. The method of example 23 wherein the second branch vessel is modulated before the main vessel is modulated.

25. The method of any one of examples 22-24 wherein modulating nerve tissue within an anatomical region extending circumferentially around the main vessel includes using the neuromodulation element to preferentially modulate nerve tissue within the anatomical region extending circumferentially around the distalmost portion of the main vessel relative to nerve tissue within an anatomical region extending circumferentially around a proximal-most portion of the main vessel and relative to nerve tissue within an anatomical region extending circumferentially around a middle portion of the main vessel between the proximal-most and distalmost portions of the main vessel.

26. The method of any one of examples 22-24 wherein modulating nerve tissue within an anatomical region extending circumferentially around the main vessel includes using the neuromodulation element to preferentially modulate nerve tissue within the anatomical region extending circumferentially around the distalmost third of the main vessel.

27. The method of any one of examples 22-26 wherein modulating nerve tissue within an anatomical region extending circumferentially around the branch vessel includes using the neuromodulation element to form between two and four lesions extending through a wall of the main vessel into the anatomical region extending circumferentially around the branch vessel.

28. The method of any one of examples 22-27 wherein modulating nerve tissue within an anatomical region extending circumferentially around the branch vessel includes modulating nerve tissue with a first power level, and wherein modulating nerve tissue within an anatomical region extending circumferentially around the main vessel includes modulating nerve tissue with a second power level greater than the first power level.

29. The method of any one of examples 22-28 wherein the method reduces sympathetic neural activity in the human patient.

30. The method of any one of examples 22-28 wherein the method reduces norepinephrine spillover in the human patient.

31. The method of any one of examples 22-28 wherein modulating nerve tissue within an anatomical region extending circumferentially around the branch vessel includes modulating nerve tissue extending circumferentially around a primary trunk segment of the branch vessel.

32. The method of any one of examples 22-28 wherein modulating nerve tissue within an anatomical region extending circumferentially around the branch vessel includes forming between about 2 lesions and about 4 lesions through an inner wall of the branch vessel from about 1 mm to about 6 mm distal to the bifurcation.

33. The method of any one of examples 22-28 wherein modulating nerve tissue within an anatomical region extending circumferentially around the branch vessel includes forming between about 2 lesions and about 4 lesions through an inner wall of the branch vessel in a region of the branch vessel from approximately 4 mm to approximately 10 mm distal to the bifurcation.

34. The method of any one of examples 22-28 wherein modulating nerve tissue within an anatomical region extending circumferentially around the branch vessel includes forming between about 2 lesions and about 4 lesions through an inner wall of the branch vessel in a region of the branch vessel from approximately 3 mm to approximately 9 mm distal to the bifurcation.

35. The method of any one of examples 22-28 wherein modulating nerve tissue within an anatomical region extending circumferentially around the branch vessel includes forming between about 2 lesions and about 4 lesions through an inner wall of the branch vessel, and wherein a distalmost lesion is at least about 9 mm distal to the bifurcation.

36. A method for treating a human patient diagnosed with a measurable physiological parameter associated with systemic sympathetic overactivity or hyperactivity, comprising:
neuromodulating renal nerve tissue within an anatomical region extending circumferentially around a branch renal vessel, wherein the branch renal vessel is located distal to a bifurcation in a main renal artery of the human patient;
neuromodulating renal nerve tissue within an anatomical region extending circumferentially around the main renal artery of the human patient; and
wherein the method reduces sympathetic neural activity in the human patient.

37. The method of example 36 wherein the measurable physiological parameter is elevated blood pressure.

38. The method of example 36 or example 37 wherein the human patient is hypertensive.

39. The method of any one of examples 36-38 wherein the method reduces norepinephrine spillover in the human patient.

40. The method of example 36 wherein neuromodulating renal nerve tissue within an anatomical region extending circumferentially around a branch renal vessel includes modulating nerve tissue extending circumferentially around a primary trunk segment of the branch renal vessel.

41. The method of example 40 wherein the primary trunk segment is about 1 mm to about 6 mm distal to the bifurcation.

42. The method of example 40 or example 41 wherein modulating nerve tissue extending circumferentially around a primary trunk segment of the branch renal vessel includes forming between about 2 lesions and about 4 lesions in a spiral-shaped pattern through an inner wall of the branch renal vessel at the primary trunk segment

43. The method of example 36 wherein neuromodulating renal nerve tissue within an anatomical region extending circumferentially around a branch renal vessel includes modulating nerve tissue extending circumferentially around a segment of the branch renal vessel between about 3 mm to about 12 mm distal to the bifurcation.

44. A method, comprising:
intravascularly positioning a neuromodulation element of a catheter within renal vasculature of a human patient, the renal vasculature including—
   a main vessel directly connected to an aorta of the patient and extending distally toward a kidney,
   a bifurcation at a distal end of the main vessel, and
   a branch vessel distal to the bifurcation;
modulating nerve tissue within an anatomical region extending circumferentially around the branch vessel along a longitudinal length of the branch vessel between about 1 mm to about 7 mm distal to the bifurcation.

45. The method of example 44 wherein intravascularly positioning a neuromodulation element of a catheter within renal vasculature of a human patient includes intravascularly positioning the neuromodulation element of the catheter within a first branch vessel, and wherein the method further includes intravascularly positioning the neuromodulation element of the catheter within a second branch vessel.

46. The method of example 45, further comprising modulating nerve tissue within an anatomical region extending circumferentially around the second branch vessel along a longitudinal length of the second branch vessel between about 1 mm to about 7 mm distal to the bifurcation.

47. A method, comprising:
intravascularly positioning a neuromodulation element of a catheter within renal vasculature of a human patient, the renal vasculature including—
   a main vessel directly connected to an aorta of the patient and extending distally toward a kidney,
   a bifurcation at a distal end of the main vessel, and
   a branch vessel distal to the bifurcation;
modulating nerve tissue within an anatomical region extending circumferentially around the branch vessel along a longitudinal length of the branch vessel between about 3 mm to about 12 mm distal to the bifurcation.

48. The method of example 47 wherein modulating nerve tissue includes forming between about 2 lesions and about 4 lesions through an inner wall of the branch vessel, and wherein a distalmost lesion is at least about 9 mm distal to the bifurcation.

49. The method of example 47 wherein modulating nerve tissue includes forming between about 2 lesions and about 4 lesions through an inner wall of the branch vessel, and wherein a distalmost lesion is at least about 7 mm distal to the bifurcation.

50. The method of example 47 wherein modulating nerve tissue includes forming between about 2 lesions and about 4 lesions through an inner wall of the branch vessel, and wherein a distalmost lesion is at least about 5 mm distal to the bifurcation.

51. A device configured to perform any of the methods of the preceding examples 1-50.

### CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments may also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

The methods disclosed herein include and encompass, in addition to methods of practicing the present technology (e.g., methods of making and using the disclosed devices and systems), methods of instructing others to practice the present technology. For example, a method in accordance with a particular embodiment includes intravascularly advancing an elongate shaft of a catheter to renal vasculature of a human patient, locating a neuromodulation element of the catheter within a distalmost portion of a main vessel of the renal vasculature, and modulating nerve tissue within an anatomical region extending circumferentially around the distalmost portion of the main vessel via the neuromodulation element. A method in accordance with another embodiment includes instructing such a method.

The headings provided herein are for convenience only and should not be construed as limiting the subject matter disclosed. As used herein, the terms “distal” and “proximal” define a position or direction with respect to a clinician or a clinician's control device (e.g., a handle of a catheter). The terms “distal” and “distally” refer to a position distant from or in a direction away from a clinician or a clinician's control device. The terms “proximal” and “proximally” refer to a position near or in a direction toward a clinician or a clinician's control device. Within an un-catheterized renal artery, the terms “distal” and “distally” refer to a position distant from or in a direction away from the renal artery ostium. The terms “proximal” and “proximally” refer to a position near or in a direction toward the renal artery ostium. Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation.

Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments of the present technology.

I/we claim:

1. A method, comprising:
intravascularly positioning a neuromodulation element of a catheter within renal vasculature of a human patient, the renal vasculature including—
a main vessel directly connected to an aorta of the patient and extending distally toward a kidney,
a bifurcation at a distal end of the main vessel, and
a branch vessel distal to the bifurcation;
modulating nerve tissue within an anatomical region extending circumferentially around the branch vessel along a longitudinal length of the branch vessel between about 1 mm to about 7 mm distal to the bifurcation.

2. The method of claim 1 wherein intravascularly positioning a neuromodulation element of a catheter within renal vasculature of a human patient includes intravascularly positioning the neuromodulation element of the catheter within a first branch vessel, and wherein the method further includes intravascularly positioning the neuromodulation element of the catheter within a second branch vessel.

3. The method of claim 2, further comprising modulating nerve tissue within an anatomical region extending circumferentially around the second branch vessel along a longitudinal length of the second branch vessel between about 1 mm to about 7 mm distal to the bifurcation.

4. A method, comprising:
intravascularly positioning a neuromodulation element of a catheter within renal vasculature of a human patient, the renal vasculature including—
a main vessel directly connected to an aorta of the patient and extending distally toward a kidney,
a bifurcation at a distal end of the main vessel, and
a branch vessel distal to the bifurcation;
ablating renal nerve tissue within an anatomical region extending circumferentially around the branch vessel along a longitudinal length of the branch vessel between about 3 mm to about 12 mm distal to the bifurcation.

5. The method of claim 4 wherein ablating renal nerve tissue includes forming between about 2 lesions and about 4 lesions through an inner wall of the branch vessel, and wherein a distalmost lesion is at least about 9 mm distal to the bifurcation.

6. The method of claim 4 wherein ablating renal nerve tissue includes forming between about 2 lesions and about 4 lesions through an inner wall of the branch vessel, and wherein a distalmost lesion is at least about 7 mm distal to the bifurcation.

7. The method of claim 4 wherein ablating renal nerve tissue includes forming between about 2 lesions and about 4 lesions through an inner wall of the branch vessel, and wherein a distalmost lesion is at least about 5 mm distal to the bifurcation.

8. A method, comprising:
intravascularly advancing an elongate shaft of a catheter to renal vasculature of a human patient, the renal vasculature including—
a main vessel directly connected to an aorta of the patient and extending distally toward a kidney, and
a bifurcation at a distal end of the main vessel;
locating a neuromodulation element of the catheter within a distalmost portion of the main vessel; and
ablating nerve tissue within an anatomical region extending circumferentially around the distalmost portion of the main vessel via the neuromodulation element.

9. The method of claim 8 wherein:
the main vessel has a longitudinal axis extending from the aorta to the bifurcation;
ablating nerve tissue within the anatomical region extending circumferentially around the distalmost portion of the main vessel includes using the neuromodulation element to form one or more lesions extending through a wall of the main vessel into the anatomical region extending circumferentially around the distalmost portion of the main vessel; and
the one or more lesions collectively are—
circumferentially continuous within the anatomical region along a plane perpendicular to a portion of the longitudinal axis extending through the distalmost portion of the main vessel, and
circumferentially discontinuous at the wall of the main vessel along all planes perpendicular to the portion of the longitudinal axis extending through the distalmost portion of the main vessel.

10. The method of claim 8 wherein the main vessel is stented, and wherein locating a neuromodulation element of the catheter within a distalmost portion of the main vessel includes locating the neuromodulation element distal to a stent.

11. The method of claim 8 wherein ablating nerve tissue within the anatomical region extending circumferentially around the distalmost portion of the main vessel includes using the neuromodulation element to preferentially ablate nerve tissue within the anatomical region extending circumferentially around the distalmost portion of the main vessel relative to nerve tissue within an anatomical region extending circumferentially around a proximal-most portion of the main vessel and relative to nerve tissue within an anatomical region extending circumferentially around a middle portion of the main vessel between the proximal-most and distalmost portions of the main vessel.

12. The method of claim 8 wherein:
the catheter, the shaft, and the neuromodulation element are a first catheter, a first shaft, and a first neuromodulation element, respectively; and
the method further comprises—
withdrawing the first catheter from the patient,
intravascularly advancing an elongate second shaft of a second catheter to the renal vasculature,
locating a second neuromodulation element of the second catheter within a branch vessel of the renal vasculature distal to the bifurcation, and
ablating nerve tissue within an anatomical region extending circumferentially around the branch vessel via the second neuromodulation element.

13. The method of claim 12, further comprising measuring a degree of neuromodulation achieved using the first neuromodulation element to ablate nerve tissue within the anatomical region extending circumferentially around the distalmost portion of the main vessel, and wherein locating the second neuromodulation element and ablating nerve tissue within the anatomical region extending circumferentially around the branch vessel includes locating the second neuromodulation element and using the second neuromodulation element to ablate nerve tissue within the anatomical region extending circumferentially around the branch vessel in response to an insufficiency of the degree of neuromodulation.

14. The method of claim 8 wherein:
advancing the shaft includes advancing the shaft while the neuromodulation element is in a low-profile delivery state; and
the method further comprises transforming the neuromodulation element between the low-profile delivery state and an expanded treatment state after locating the neuromodulation element within the distalmost portion of the main vessel and before ablating nerve tissue within the anatomical region extending circumferentially around the distalmost portion of the main vessel.

15. The method of claim 14 wherein:
the neuromodulation element includes a balloon; and
transforming the neuromodulation element includes inflating the balloon.

16. The method of claim 14 wherein:
the neuromodulation element includes an elongate support structure carrying a plurality of electrodes, the support structure having a helical form when unconstrained;
advancing the shaft includes advancing the shaft while the support structure is constrained; and
transforming the neuromodulation element includes reducing constraint on the support structure such that the support structure moves toward having the helical form.

17. The method of claim 14 wherein:
the neuromodulation element includes an elongate electrode having a helical form when unconstrained;
advancing the shaft includes advancing the shaft while the electrode is constrained; and
transforming the neuromodulation element includes reducing constraint on the electrode such that the electrode moves toward having the helical form.

18. The method of claim 8 wherein:
the neuromodulation element is a first neuromodulation element; and
the method further comprises—
locating a second neuromodulation element of the catheter within a branch vessel of the renal vasculature distal to the bifurcation, and
ablating nerve tissue within an anatomical region extending circumferentially around the branch vessel after locating the second neuromodulation element.

19. The method of claim 18 wherein:
advancing the shaft includes advancing the shaft while the second neuromodulation element is in a low-profile delivery state; and
the method further comprises transforming the second neuromodulation element between the low-profile delivery state and an expanded treatment state after locating the second neuromodulation element and modulating nerve tissue within the anatomical region extending circumferentially around the branch vessel.

20. The method of claim 18 wherein:
the second neuromodulation element includes a balloon; and
transforming the second neuromodulation element includes inflating the balloon.

21. The method of claim 18 wherein:
the second neuromodulation element includes an elongate support structure carrying a plurality of electrodes, the support structure having a helical form when unconstrained;
advancing the shaft includes advancing the shaft while the support structure is constrained; and
transforming the second neuromodulation element includes reducing constraint on the support structure such that the support structure moves toward having the helical form.

22. The method of claim 18 wherein:
the second neuromodulation element includes an elongate electrode having a helical form when unconstrained;
advancing the shaft includes advancing the shaft while the electrode is constrained; and
deploying the second neuromodulation element includes reducing constraint on the electrode such that the electrode moves toward having the helical form.

23. The method of claim 18, further comprising measuring a degree of neuromodulation achieved using the first neuromodulation element to ablate nerve tissue within the anatomical region extending circumferentially around the distalmost portion of the main vessel, wherein locating the second neuromodulation element and ablating nerve tissue within the anatomical region extending circumferentially around the branch vessel includes locating the second neuromodulation element and ablating nerve tissue within the anatomical region extending circumferentially around the branch vessel in response to an insufficiency of the degree of neuromodulation.
